(19) United States
(12) Patent Application Publication
(10) Pub. No.: US 2023/0364191 A1
(43) Pub. Date: Nov. 16, 2023

(54) COMPOSITIONS AND METHODS FOR PERSONALIZED TREATMENT OF NEURODEGENERATIVE CONDITIONS OR SIDE EFFECTS THEREOF

(71) Applicant: THE REGENTS OF THE UNIVERSITY OF COLORADO, A BODY CORPORATE, Denver, CO (US)

(72) Inventors: Huntington Potter, Denver, CO (US); Timothy Boyd, Johnstown, CO (US); Stefan Sillau, Denver, CO (US)

(21) Appl. No.: 18/227,184

(22) Filed: Jul. 27, 2023

Related U.S. Application Data
(63) Continuation of application No. PCT/US2022/016219, filed on Feb. 11, 2022.
(60) Provisional application No. 63/148,824, filed on Feb. 12, 2021.

(51) Int. Cl. A61K 38/19; G01N 33/68

(52) U.S. Cl. CPC A61K 38/193; G01N 33/6896; G01N 33/6827; G01N 33/6863

(57) ABSTRACT

Embodiments of the instant disclosure relate to novel methods for treatment of a neurodegenerative disease or condition. In certain embodiments, methods of alleviating a neurodegenerative disease in a subject include administering an effective concentration of granulocyte macrophage colony stimulating factor (GM-CSF); monitoring an absolute number of at least one of leukocytes, concentration of at least one inflammatory cytokine, concentration of at least one neurodegenerative condition biomarker, or a combination thereof in the subject; and adjusting GM-CSF treatment regimens based the level of at least one of these parameters. In some embodiments, methods of alleviating a neurodegenerative condition in a subject can further include performing at least one cognition assessment test before and after GM-CSF treatment and adjusting the GM-CSF treatment regimen based on observations in the cognitive state of the subject before and after administration of GM-CSF.

### PRIORITY

This application is a U.S. Continuation Application which claims priority to International Application PCT/US2022/016219, filed Feb. 11, 2022, which claims priority to U.S. Provisional Application No. 63/148,824 filed Feb. 12, 2021. These applications are incorporated herein by reference in their entirety for all purposes.

### FIELD

Embodiments of the instant disclosure relate to methods for treating a neurodegenerative condition. In certain embodiments, compositions, and methods for improving cognition by administering granulocyte macrophage colony stimulating factor (GM-CSF) and monitoring parameters in blood samples are disclosed.

### BACKGROUND

Neurodegenerative conditions causing cognitive disorders encompasses a range of conditions such as Alzheimer's disease (AD), Parkinson's disease (PD), Huntington's disease (HD) and the like. These conditions can occur when neurons in the central or peripheral nervous system progressively lose function and eventually die. Currently available treatments only help relieve some of the physical or certain cognitive symptoms associated with neurodegenerative diseases and their benefits are transient. Further, these treatments are only effective for about half of the patients that take them. Therefore, there is a need to develop new methods for treating these conditions to prevent or reverse neurodegenerative condition-related cognitive decline. A need exists to find additional treatment regimens that reduce inflammation and lead to improved cognition in subjects having neurodegenerative disorders.

### SUMMARY

Embodiments of the instant disclosure relate to novel compositions and methods for treating one or more neurodegenerative conditions. In certain embodiments, methods for treating a neurodegenerative condition in a subject can include administering granulocyte macrophage colony stimulating factor (GM-CSF) to the subject. In some embodiments, compositions including, but not limited to, GM-CSF can be used in a predetermined treatment regimen while monitoring the subject in order to provide improved, individualized treatment regimens for optimal outcomes. In accordance with these embodiments, these regimens can be monitored and adjusted in order to provide improved cognition and/or reduced neuronal damage. In certain embodiments, a subject having a neurodegenerative condition can be treated with a GM-CSF treatment regimen and absolute numbers of at least one leukocyte cell and/or concentration of at least one inflammatory marker can be monitored before, during and/or after the GM-CSF treatment regimen. In other embodiments, concentrations of at least one neurodegenerative condition-linked biomarker can be monitored before, during or after the GM-CSF treatment regimen. In some other embodiments, a ratio of albumin to globulin can be monitored before, during or after the GM-CSF treatment regimen. In some embodiments, absolute numbers of at least one leukocyte cell, concentration of at least one inflammatory marker, ratio of albumin to globulin, and/or concentrations of at least one neurodegenerative condition-linked biomarker can be monitored before, during and/or after GM-CSF treatment wherein the GM-CSF treatment regimen can be adjusted based on observations of one or more of these parameters. In other embodiments, at least one cognition test can be administered to the subject having the neurodegenerative disorder before, during and/or after treating the subject with a GM-CSF treatment regimen accessing the outcome of the at least one cognition test and optimizing the subject's treatment regimen to improve cognition in the subject.

In some embodiments, a subject can undergo an initial GM-CSF dosing regimen for about four or more days per week for a time of up to about 2 to about 4 weeks. In certain embodiments, the above referenced parameters can be assessed before, during or after this initial GM-CSF dosing regimen. In some embodiments, the initial GM-CSF dosing regimen can be analyzed for effect on cognition using a cognition exam and/or assessed for concentration or presence of at least one inflammatory marker in a t least one blood sample from the subject. In accordance with these embodiments, when cognition is improved and levels of leukocytes are maintained, GM-CSF dosing regimens can be continued.

In some embodiments, the initial GM-CSF dosing regimen can continue when the absolute number of at least one leukocyte (e.g., monocyte, lymphocyte, neutrophil), the concentration of at least one inflammatory marker (e.g., cytokine), the concentration of at least one neurodegenerative condition-related biomarker, or the combination thereof in a subject is modified after administration of a GM-CSF-containing composition for at least five days per week for up to about three weeks. In some embodiments, an absolute number of at least one leukocyte can be an absolute number of monocytes, an absolute number of lymphocytes, an absolute number of neutrophils, or a combination thereof. In other embodiments, the at least one inflammatory marker can include at least one cytokine. In accordance with these embodiments, at least one cytokine can include, but is not limited to, interleukin (IL)-2, IL-6, IL-8, IL-10, Tumor Necrosis Factor (TNF)-alpha, other proinflammatory cytokines or a combination thereof. In other embodiments, the at least one neurodegenerative disorder-related biomarker can include, but is not limited to, at least one Alzheimer's-related biomarker. In certain embodiments, Alzheimer's-related biomarkers can include, but are not limited to, Amyloid beta, Tau, ubiquitin C-terminal hydrolase L1 (UCH-L1), or a combination thereof.

In some embodiments, a GM-CSF dosing regimen can be stopped when the absolute number of lymphocytes is 15% or greater and/or the absolute number of monocytes is 30% or greater compared to a baseline level in a blood sample from the subject collected before administration of an initial GM-CSF dosing regimen. In some embodiments, a GM-CSF dosing regimen can continue when the absolute number of monocytes is less than about 30% and when the absolute number of lymphocytes is less than about 15% compared to a baseline level in a blood sample from the subject collected before administration of a GM-CSF-containing composition dosing regimen. In some embodiments, the concentration of Tau and/or UCH-L1 can be measured in a subject having a neurodegenerative disorder (e.g. Alzheimer's) before, during and/or after a GM-CSF-containing composition dosing regimen. In accordance with these embodiments, if the concentration of Tau and/or UCH-L1 is greater than about 10% relative to a baseline level in a blood sample from the subject collected before administration of a GM-CSF-containing composition, then administration of GM-CSF can and possibly, should continue. In other embodiments, if the level of Tau and/or UCH-L1 is stabilized within about 10% of a baseline level in a blood sample from the subject collected before administration of a GM-CSF-containing composition, then the GM-CSF regimen can be stopped for a period of time or altogether. In some embodiments, a GM-CSF-containing composition dosing regimen can be stopped if the concentration of UCH-L1 and/or concentration of Tau in a subject's sample increases to about 30% or greater compared to a baseline level in the subject. In accordance with these embodiments, clinical and cognitive analyses can be recommended to be performed on a subject having these levels of UCH-L1 and/or Tau. In certain embodiments, where a subject's GM-CSF-containing composition dosing regimen is closely monitored as disclosed herein, a subject can experience improved cognition while reducing exposure to unnecessary levels of GM-CSF and/or conserving resources.

In some embodiments, GM-CSF used in methods disclosed herein can be formulated in a pharmaceutical composition, which can further include at least one pharmaceutically acceptable carrier or excipient. In other embodiments, GM-CSF or compositions containing GM-CSF thereof can be administered to a subject by any method known in the art. In certain embodiments, delivery can include, but is not limited to administering GM-CSF by controlled dosing using timed-release formulations for example, by microparticle, by beads, dissolving strips, or the like. In other embodiments, administering GM-CSF to a subject can include an initial burst and then a more steady-state administration. In certain embodiments, GM-CSF can be administered to a subject by a parenteral route or orally or a combination depending on the subject and condition of the subject to be treated.

In some embodiments, a subject contemplated to be treated by compositions and methods disclosed herein can be a human subject having a neurodegenerative condition, an infection causing neurodegenerative side effects, or a neurodegenerative disease. In other embodiments, a neurodegenerative condition disclosed herein can include, but is not limited to, Alzheimer's disease, frontotemporal dementia, vascular dementia, viral infection, or a combination thereof. In some embodiments, a subject can have mild stage Alzheimer's disease, moderate stage Alzheimer's disease, or severe Alzheimer's disease.

In certain embodiments, methods for monitoring GM-CSF treatment in a subject can include one or more of the following: a) performing at least one cognitive state exam; for example, before treatment for a baseline level; b) collecting and analyzing at least one blood sample; for example, before treatment for 1) a baseline level analysis of leukocyte levels by measuring an absolute number of at least one leukocyte (e.g. monocyte, lymphocyte and/or neutrophil); 2) a baseline level of concentration of at least one inflammatory marker (e.g. cytokine); 3) a baseline level of concentration of at least one neurodegenerative disorder-related biomarker, or a combination thereof in the baseline blood sample. In accordance with these embodiments, the subject can then be treated with a GM-CSF dosing regimen and monitored to assess additional treatment plans for the subject. In some embodiments, the GM-CSF dosing regimen can include GM-CSF treatments of at least about five days per week for up to about three weeks. In accordance with these embodiments, treating a subject with a composition containing GM-CSF or GM-CSF supplement can include treating the subject one time daily with a dose of about 50 μg/m²/day to about 500 μg/m²/day; or about 100 μg/m²/day to about 300 μg/m²/day; or about 125 μg/m²/day to about 250 μg/m²/day.

In other embodiments, follow-up analysis of cognition and/or blood sample analysis can be performed during and/or after a GM-CSF treatment regimen. In accordance with these embodiments, follow-up analysis can include, but is not limited to, performing at least one addition cognition test and/or obtaining at least one blood sample and measuring absolute number of at least one leukocyte, measuring concentration of at least one inflammatory marker (e.g. cytokine), measuring concentration of at least one neurodegenerative condition-related biomarker, or a combination thereof in the follow-up blood sample(s). In certain embodiments, baseline and subsequent blood samples can be compared and/or cognition testing results compared in order to assess whether the GM-CSF treatment regimen should be adjusted for optimum results, reduce a subject's drug exposure and/or conserve expenses.

In some embodiments, the at least one cognitive state exam can include, but are not limited to, a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), other cognition test or a combination thereof. In some embodiments, the at least one cognitive state exam or cognition test can be a Mini-Mental State Exam (MMSE).

In certain embodiments, kits for treating and/or analyzing a subject having a neurodegenerative condition are contemplated. In some embodiments, kits disclosed herein can include a GM-CSF-containing composition and at least one container. In other embodiments, kits disclosed herein can further include at least one container and/or device for use in collecting a baseline blood sample, analyzing a baseline blood sample, collecting a follow-up blood sample, analyzing a follow-up blood sample, or a combination thereof.

### BRIEF DESCRIPTION OF THE DRAWINGS

FIGS. 1A-1E illustrate examples of markers of leukocytes in GM-CSF (e.g. sargramostim) treated human subjects compared to placebo-treated human subjects in accordance with certain aspects of the present disclosure. FIGS. 1A-1C show graphs depicting absolute numbers of monocytes (FIG. 1A), lymphocytes (FIG. 1B), and neutrophils (FIG. 1C) during the treatment phase in the sargramostim group compared to the placebo group. FIG. 1D shows a representative image of an exemplary Meso-Scale Discovery method used herein to determine changes in plasma inflammatory cytokines with sargramostim treatment. FIG. 1E shows a graph depicting the ratio of plasma albumin to globulin used in assessment of inflammation and its acute phase response.

FIGS. 2A-2C illustrate examples of Mini-Mental State Exam (MMSE) scores in sargramostim-treated human subjects compared to placebo-treated human subjects in accordance with certain aspects of the present disclosure. FIG. 2A shows a graph depicting a mixed model analysis of MMSE data from sargramostim-treated and placebo-treated participants. FIG. 2B shows a bar graph depicting improvements in total MMSE score in sargramostim-treated and placebo-treated participants at the end of treatment (EOT), at the 45-day follow-up visit (FU1), and at the 90-day follow-up visit (FU2). FIG. 2C shows a bar graph depicting mean MMSE scores calculated and graphed as a delta distribution for the sargramostim-treated and placebo-treated participants at EOT.

FIGS. 3A and 3B illustrate examples of ADAS-Cog-13 Score and its Memory. Domain Subscale in sargramostim-treated human subjects compared to placebo-treated human subjects in accordance with certain aspects of the present disclosure. FIG. 3A shows a graph depicting changes in ADAS-Cog13 scores between sargramostim-treated participants and placebo-treated participants during the treatment phase and up to 100 days thereafter. FIG. 3B shows a graph depicting memory domain subscales of ADAS-Cog13 (ADAS delayed word recall+ADAS word recognition+ADAS orientation+ADAS word recall average) of sargramostim-treated participants and placebo-treated participants during the treatment phase and up to 100 days thereafter.

FIGS. 4A-4C illustrate examples of neurodegenerative condition-linked markers in sargramostim-treated human subjects compared to placebo-treated human subjects in accordance with certain aspects of the present disclosure. FIG. 4A shows a graph depicting amyloid beta (Aβ)40 in the plasma of sargramostim-treated and placebo-treated participants during the treatment phase and up to 90 days thereafter. FIG. 4B shows a graph depicting total tau levels in the plasma of sargramostim-treated and placebo-treated participants during the treatment phase and up to 90 days thereafter. FIG. 4C shows a graph depicting plasma ubiquitin C-terminal hydrolase L1 (UCH-L1) in the plasma of sargramostim-treated and placebo-treated participants during the treatment phase and up to 90 days thereafter.

FIGS. 5A-5E illustrate examples of correlations among and between changes in behavior and of biomarkers of neuropathology in sargramostim-treated human subjects compared to placebo-treated human subjects in accordance with certain aspects of the present disclosure. FIG. 5A shows a graph depicting a Pearson correlation between the change in MMSE and change in absolute neutrophil counts at end of treatment (EOT) in sargramostim-treated participants. FIG. 5B shows a graph depicting a Pearson correlation between the change in MMSE and change in absolute lymphocyte counts at EOT in sargramostim-treated participants. FIG. 5C shows a graph depicting a Pearson correlation between the change in plasma glial fibrillary acidic protein and plasma neurofilament light at EOT in sargramostim-treated participants. FIGS. 5D-5E show graphs depicting Pearson correlations between changes in MMSE and Activities of Daily Living at EOT (FIG. 5D) and at the 45-day follow-up visit (FU1; FIG. 5E) in sargramostim-treated participants.

FIG. 6 illustrates a graph of ADCS-ADL scores in sargramostim-treated human subjects compared to placebo-treated human subjects in accordance with certain aspects of the present disclosure.

FIG. 7 illustrates a graph of CDR-SB measurements in sargramostim-treated human subjects compared to placebo-treated human subjects in accordance with certain aspects of the present disclosure.

FIG. 8 illustrates a graph of TRAILS-A measurements in sargramostim-treated human subjects compared to placebo-treated human subjects in accordance with certain aspects of the present disclosure.

FIGS. 9A-9B illustrate examples of coronal tissue after intrahippocampal injection of GM-CSF in an animal model of a neurodegenerative condition in accordance with certain aspects of the present disclosure. FIG. 9A shows a representative image of a coronal tissue stained with MabTech α-Aβ/Alexa 546. FIG. 9B shows a bar graph depicting four plaque parameters (Area, Perimeter, Feret Diameter, and Integrated Density) measured in coronal tissue samples harvested from GM-CSF-treated and aCSF treated mice.

FIGS. 10A-10F illustrate examples of behavioral and pathology analysis following subcutaneous GM-CSF injections in an animal model of neurodegenerative disease in accordance with certain aspects of the present disclosure. FIGS. 10A-10D show graphs depicting errors made by Tg control mice, NT control mice, GM-CSF-treated Tg mice, and GM-CSF-treated NT mice in working memory trials T4 and T5 of a Radial Arm Water Maze in Block 1 (FIG. 10A) and Block 2 (FIG. 10B) of testing and over 4 days of T4 (FIG. 10C) and T5 (FIG. 10D) testing. FIG. 10E shows a graph depicting a percentage of amyloid burden in mouse hippocampus (H) and entorhinal cortex (EC) in GM-CSF- and saline-treated mice. FIG. 10F shows a graph depicting a percentage of Iba1 burden in mouse hippocampus (H) and entorhinal cortex (EC) in GM-CSF- and saline-treated mice.

FIG. 11 illustrates an example of synaptophysin immunostaining in a subcutaneous GM-CSF-injected animal model of neurodegenerative condition in accordance with certain aspects of the present disclosure.

FIG. 12 illustrates an example of neuropsychological performance in hematopoietic cell transplant (HCT) human recipients receiving GM-CSF+G-CSF versus G-CSF only in accordance with certain aspects of the present disclosure.

FIG. 13 illustrates a schematic depicting a design for a two-center, randomized, three-week-long, double-blind, placebo-controlled, study of sargramostim in patients with mild to moderate Alzheimer's disease (AD).

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” and “patient” are used interchangeably herein and refer to both human and nonhuman animals as applicable. The term “nonhuman animals” of the disclosure includes all vertebrates, e.g., mammals and non-mammals, such as nonhuman primates, horses, sheep, dog, cat, and the like. In some embodiments, the subject is a human. In other embodiments, the subject is a human in need of treatment for a neurodegenerative disorder.

As used herein, “treatment,” “therapy” “treatment regimen” and/or “therapy regimen” can refer to the clinical intervention made in response to a condition, disease, disorder, or physiological condition manifested by a subject or to which a subject can be susceptible. The aim of treatment includes the alleviation or prevention of symptoms, slowing or stopping the progression or worsening of a condition, disease, disorder and/or the remission of the condition, disease, or disorder.

As used herein, “prevent” or “prevention” refers to eliminating or delaying the onset of a particular disease, disorder, or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

Unless otherwise defined, all technical terms used herein have the same meaning as commonly understood by one of ordinary skill in the art to which this disclosure belongs.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Embodiments of the instant disclosure relate to novel methods for treating a neurodegenerative condition. In accordance with these embodiments, a neurodegenerative condition can include, but is not limited to Alzheimer's disease (AD), Parkinson's disease (PD), Huntington's disease (HD), frontotemporal dementia, vascular dementia, a viral infection causing a neurodegenerative condition, and the like.

Embodiments disclosed herein concern treating one or more neurodegenerative conditions for improved outcome to alleviate, alleviate symptoms of, reduce advancement of, or eliminate the neurodegenerative condition. In certain embodiments, a subject having a neurodegenerative condition can be treated using compositions disclosed herein where the treatment regimen is monitored to assess improved cognition and/or reduced inflammation. In certain embodiments, methods for treating a neurodegenerative condition in a subject can include administering GM-CSF or a GM-CSF-containing composition in a predetermined regimen and monitoring the subject in order to provide improved, individualized treatment regiments for optimal outcomes including, but not limited to, improved cognition and/or reduced neuronal damage. In accordance with these embodiments, a subject having a neurodegenerative condition can be treated with a GM-CSF or GM-CSF-containing composition treatment regimen and absolute numbers of at least one leukocyte cell and/or concentration of at least one inflammatory marker can be monitored before, during and/or after the treatment regimen. In other embodiments, concentrations of at least one neurodegenerative condition-linked marker can be monitored before, during and/or after the treatment regimen. In some embodiments, absolute numbers of at least one leukocyte cell, concentration of at least one inflammatory marker, ratio of albumin to globulin, and/or concentrations of at least one neurodegenerative condition-linked marker can be monitored before, during and/or after a GM-CSF treatment or treatment regimen where the GM-CSF treatment regimen can be adjusted based on observations of one or more of these parameters. In other embodiments, at least one cognition test can be administered to the subject having the neurodegenerative condition or disorder before, during and/or after treating the subject with a GM-CSF treatment regimen in order to optimize a subject's treatment regimen to improve cognition and reduce side effects of the neurodegenerative condition in the subject.

In some embodiments, a subject having a neurodegenerative condition can undergo an initial GM-CSF dosing regimen for about four or more days per week for up about 2 to about 4 weeks or longer. In certain embodiments, the above referenced parameters can be assessed before, during and/or after this initial GM-CSF dosing regimen where baseline parameters can be compared to the same or different parameters during and/or after GM-CSF treatment. In some embodiments, the initial GM-CSF dosing regimen can be analyzed for effect on cognition using a cognition exam and/or assessed for concentration of, or presence of leukocytes in a blood sample from the subject. In accordance with these embodiments, when cognition is improved and levels of leukocytes are maintained, additional GM-CSF dosing regimens can be continued; and optionally, reassessed after a predetermined period. In some embodiments, the initial GM-CSF dosing regimen can be analyzed for effect on cognition using a cognition exam and/or assessed for the ratio of albumin to globulin in a blood sample from the subject. In accordance with these embodiments, when cognition is improved and the ratio of albumin to globulin is maintained, additional GM-CSF dosing regimens can be continued; and optionally, reassessed after a predetermined period.

In some embodiments, the initial GM-CSF dosing regimen or a subsequent GM-CSF dosing regimen can continue when the absolute number of at least one leukocyte (e.g. monocyte, lymphocyte, neutrophil), the concentration of at least one inflammatory marker (e.g. cytokine), the concentration of at least one neurodegenerative condition-related biomarker, the ratio of albumin to globulin, or the combination thereof in a subject is improved (e.g. reduced or maintained) after administration of GM-CSF for at least four to five days per week for up to about three weeks. In certain embodiments, an absolute number of at least one leukocyte can be an absolute number of monocytes, an absolute number of lymphocytes, an absolute number of neutrophils, or a combination thereof which are indicative of immune-related responses in a subject having a neurodegenerative disorder. In some embodiments, the at least one inflammatory marker can include at least one cytokine. In accordance with these embodiments, at least one cytokine can include, but is not limited to, interleukin (IL)-2, IL-6, IL-8, IL-10, Tumor Necrosis Factor (TNF)-alpha, other proinflammatory cytokines or a combination thereof. In other embodiments, the at least one neurodegenerative disorder-related marker can include, but is not limited to, at least one Alzheimer-related biomarker or the like such as at least one neurodegenerative condition-related marker. In some embodiments, Alzheimer-related biomarkers can include, but are not limited to, Amyloid beta, Tau, ubiquitin C-terminal hydrolase L1 (UCH-L1), or a combination thereof.

In some embodiments, a GM-CSF dosing or treatment regimen in a subject being treated can be stopped or should be stopped when the absolute number of lymphocytes is 15% or greater and/or the absolute number of monocytes is 30% or greater compared to a baseline level in a subject's blood sample collected before administration of an initial GM-CSF dosing regimen. In accordance with these embodiments, the GM-CSF dosing regimen can be stopped for a predetermined number of days such as about 30 days, about 40 days, about 50 days or more depending on the subject and whether the leukocyte numbers are reduced once treatment is ceased. In some embodiments, GM-CSF treatment can be stopped for 45 days and if deemed appropriate, commenced with continued monitoring. In accordance with these embodiments, a GM-CSF dosing regimen can continue or resume when the absolute number of monocytes is less than 30% and/or when the absolute number of lymphocytes is less than 15% compared to a baseline level in a subject's blood sample collected before administration of a GM-CSF dosing regimen. In certain embodiments, GM-CSF treatment regimens can be started and stopped to optimize treatment at intervals based on whether the absolute number of monocytes is less than 30% and/or when the absolute number of lymphocytes is less than 15% compared to a baseline level in a subject's blood sample collected before administration of a GM-CSF dosing regimen.

In some embodiments, the concentration of Tau and/or UCH-L1 can be measured in a subject having a neurodegenerative disorder (e.g. Alzheimer's) before, during and/or after a GM-CSF dosing regimen. In accordance with these embodiments, if the concentration of Tau and/or UCH-L1 is greater than 10% compared to a baseline level in a subject's blood sample collected before administration of a GM-CSF dosing regimen then administration of GM-CSF can or should continue. In other embodiments, if the level of Tau and/or UCH-L1 is stabilized to within about 15% or about 10% or about 5% higher compared to a baseline level in a subject's blood sample collected before administration of a GM-CSF dosing regimen then the GM-CSF regimen can be stopped for a period of time or altogether. In some embodiments, a GM-CSF dosing regimen can be and likely should be stopped if the concentration of UCH-L1 and/or concentration of Tau in a subject's sample increases to 30% or greater during treatment compared to a baseline level in the subject. In accordance with these embodiments, clinical and cognitive analyses can be recommended to be performed on a subject having these levels of UCH-L1 and/or Tau. In certain embodiments, where a subject's GM-CSF dosing regimen is closely monitored as disclosed herein, a subject can experience improved cognition while reducing exposure to unnecessary levels of GM-CSF and/or conserving resources. In certain embodiments, GM-CSF treatment regimens can be started and stopped to optimize treatment at intervals based on whether the concentration of UCH-L1 and/or concentration of Tau in a subject's sample increases to 30% or greater during treatment compared to a baseline level in the subject and/or based on whether the absolute number of monocytes is less than 30% and/or when the absolute number of lymphocytes is less than 15% compared to a baseline level in a subject's blood sample collected before administration of a GM-CSF dosing regimen.

In some embodiments, GM-CSF used in methods herein can be formulated in a pharmaceutical composition, which further includes at least one pharmaceutically acceptable carrier or excipient. In some embodiments, GM-CSF can be administered to a subject by any method acceptable for administering GM-CSF such as controlled dosing by microparticle or the like. In other embodiments, GM-CSF can be administered to a subject by a parenteral route or orally or a combination depending on the subject and condition of the subject to be treated.

In some embodiments, a subject suitable for methods described herein can be a human subject having a neurodegenerative condition. In other embodiments, a neurodegenerative condition herein can include, but is not limited to, Alzheimer's disease, frontotemporal dementia, vascular dementia, side effects of a viral infection, or a combination thereof. In some embodiments, a subject contemplated herein can have mild stage Alzheimer's disease, moderate stage Alzheimer's disease, or severe Alzheimer's disease.

In certain embodiments, methods for monitoring GM-CSF treatment in a subject can include one or more of the following: a) performing at least one cognitive state exam; for example, before treatment to obtain a baseline level; b) collecting and analyzing at least one blood sample; for example, before treatment to obtain 1) a baseline level analysis of leukocyte levels by measuring an absolute number of at least one leukocyte (e.g. monocyte, lymphocyte and/or neutrophil); 2) a baseline level of concentration of at least one inflammatory marker (e.g. cytokine), 3) a baseline level of concentration of at least one neuropathology biomarker, 4) a baseline ratio of albumin to globulin, or a combination thereof in the baseline blood sample from the subject. In accordance with these embodiments, the subject can then be treated with a GM-CSF dosing regimen and monitored to assess whether the treatment regimen requires modification. In some embodiments, a GM-CSF dosing regimen can include GM-CSF treatments for at least about five days per week, for up to about three weeks, for up to about four weeks, for up to about five weeks, or more. In accordance with these embodiments, treatments of GM-CSF can include treating the subject one time daily with a dose of about 50 μg/m²/day to about 500 μg/m²/day; or about 100 μg/m²/day to about 300 μg/m²/day; or about 125 μg/m²/day to about 250 μg/m²/day. In accordance with these embodiments, treatments of GM-CSF can include treating the subject with a daily dose of about 50 μg/m²/day to about 500 μg/m²/day intravenously over about a 1-hour to about a 24-hour period. In accordance with these embodiments, treatments of GM-CSF can include treating the subject with a daily dose of about 50 μg/m²/day to about 500 μg/m²/day intravenously over about a 1-hour, about a 2-hour, about a 4-hour, about a 6-hour, about a 12-hour, about an 18-hour, or about a 24-hour period. In accordance with these embodiments, treatments of GM-CSF can include treating the subject with a daily dose of about 50 μg/m²/day to about 500 μg/m²/day by at least one subcutaneous injection. In accordance with these embodiments, treatments of GM-CSF can include administering to the subject between at least one to at least three subcutaneous injections per day of about 50 μg/m²/day to a total of about 500 μg/m²/day GM-CSF.

In other embodiments, follow-up analysis of cognition and/or blood sample analysis can be performed during and/or after the GM-CSF treatment regimen. In accordance with these embodiments, follow-up analysis can include performing at least one addition cognition test and/or obtaining at least one blood sample and measuring absolute number of at least one leukocyte, measuring concentration of at least one inflammatory marker (e.g. cytokine), measuring concentration of at least one neurodegenerative condition-related biomarker, measuring the ratio of albumin to globulin, or a combination thereof in the follow-up blood sample(s). In certain embodiments, baseline and subsequent blood samples can be compared and/or cognition testing results compared in order to whether GM-CSF treatment regimen can be adjusted for optimum results, as well as to reduce a subject's drug exposure and/or conserve costs.

In some embodiments, if a subject's absolute leukocyte levels during treatment compared to baseline levels reaches about 20,000/ml then the GM-CSF treatment regimen can be modified. In accordance with these embodiments, modifications of the treatment regimen can include reducing the concentration of GM-CSF to about 50 μg/m²/day to about 200 μg/m²/day; or about 100 μg/m²/day to about 200 μg/m²/day; about 125 μg/m²/day to about 200 μg/m²/day; or about half of the current daily dosing regimen. In accordance with these embodiments, a subject can continue to be monitored for absolute leukocyte levels as well as other parameters disclosed herein in order to provide optimum treatment of GM-CSF to the subject.

In some embodiments, the at least one cognition test can include, but is not limited to, a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), other cognition test or a combination thereof. In certain embodiments, the at least one cognitive state exam or cognition test can be a Mini-Mental State Exam (MMSE). In some embodiments, a cognition test can include a test administered as a baseline test, a test in a control subject not having a neurodegenerative disorder and one or more tests performed during and/or after a subject has been treated with one or more GM-CSF dosing regimen.

In some certain embodiments, methods of treating a neurodegenerative condition disclosed herein can include administration of at least GM-CSF to a subject having such a condition. In other embodiments, a GM-CSF dosing regimen can be combined with other known neurodegenerative disorder treatments of a subject in need thereof.

GM-CSF is a monomeric glycosylated polypeptide signaling molecule which is typically secreted by immune cells such as macrophages, T cells, mast cells, natural killer (NK) cells, as well as normal tissue cells such as endothelial cells and fibroblasts. In bone marrow, GM-CSF can function as a leukocyte growth factor and can stimulate hematopoietic progenitor cells to differentiate into cells of the monocytic and granulocytic lineage. In addition to its growth factor function, GM-CSF can also act as an important modulator of immune responses.

In some embodiments, a GM-CSF suitable for use herein can be a native GM-CSF, a recombinant GM-CSF, or a recombinant variant of the GM-CSF. In accordance with these embodiments, a native GM-CSF can be a human GM-CSF purified native molecule according to methods known in the art. In accordance with these embodiments, a recombinant GM-CSF and/or a recombinant variant of the GM-CSF can be obtained from polynucleotides encoding GM-CSF and/or GM-CSF variants by the use of cell-free expression systems such as reticulocyte lysate-based expression systems, or by standard recombinant expression systems. In some embodiments, polynucleotides encoding GM-CSF and/or GM-CSF variants can be cloned into expression vectors and expressed using standard procedures. In other embodiments, recombinant GM-CSF and/or GM-CSF variants can be purified using known methods in the art, including but not limited to chromatography. In some embodiments, a recombinant human GM-CSF and/or a recombinant variant of the human GM-CSF can be obtained from polynucleotides encoding human GM-CSF and/or human GM-CSF variants. In certain embodiments, a GM-CSF suitable for use herein can be a native human GM-CSF, a recombinant human GM-CSF, or a recombinant variant of human GM-CSF. In some embodiments, GM-CSF suitable for use herein can be sargramostim.

In certain embodiments, GM-CSF suitable for use herein can be formulated to form a pharmaceutical composition, which can further include a pharmaceutically acceptable carrier, diluent, or excipient. In some embodiments, any of the pharmaceutical compositions to be used in the present methods can include pharmaceutically acceptable carriers, excipients, or stabilizers in the form of lyophilized formations or aqueous solutions.

In some embodiments, pharmaceutical compositions or formulations herein can be for parenteral administration, such as intravenous, intra-articular injection, or intravenously administered through an IV-delivery device, intracerebroventricular injection, intra-cisterna magna injection, intra-parenchymal injection, or a combination thereof. In some embodiments, pharmaceutical compositions or formulations herein can be for subcutaneous injection. In other embodiments, a time-release composition can be used in order to treat the subject over a predetermined period of time with reduced subject interfacing.

In certain embodiments, formulations herein suitable for parenteral administration can include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. Aqueous solutions can be suitably buffered (preferably to a pH of from 3 to 9). The preparation of suitable parenteral formulations under sterile conditions is readily accomplished by standard pharmaceutical techniques well known to those skilled in the art.

In certain embodiments, pharmaceutical compositions disclosed herein to be used for in vivo administration should be sterile. This can be readily accomplished by, for example, filtration through sterile filtration membranes. Sterile injectable solutions are generally prepared by incorporating hydrogels in the required amount in the appropriate solvent with various other ingredients enumerated above, as required, followed by filter sterilization. Generally, dispersions can be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In certain embodiments, pharmaceutical compositions disclosed herein can also include other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

In certain embodiments, any GM-CSF, for example, native GM-CSF, recombinant GM-CSF, or a recombinant variant of GM-CSF can be used for alleviating and/or treating a neurodegenerative condition. In some embodiments, methods herein can be used for alleviating one or more symptoms and/or for treating a neurodegenerative condition in a subject in need thereof of by administering a GM-CSF described herein, as well as a pharmaceutical composition having such. In some embodiments, methods disclosed herein can include administration of an effective concentration of GM-CSF or a pharmaceutical composition to a subject who needs treatment via a suitable route (e.g., intravenous infusion or oral administration) at a suitable concentration as disclosed herein. In some examples, GM-CSF or a pharmaceutical composition herein can be administered to a subject by injection through a syringe, a catheter, a trocar, a cannula, and the like.

In certain embodiments, a subject to be treated by any of the methods disclosed herein may be a subject having or suspected of having at least one neurodegenerative condition. In some embodiments, neurodegenerative conditions to be treated by methods disclosed herein can include, but are not limited to, Alzheimer's disease (AD), Parkinson's disease (PD), Huntington's disease (HD), frontotemporal dementia, vascular dementia, side effects of a viral infection, and the like. In certain embodiments, a subject to be treated by any of the methods disclosed herein can be a human subject having or suspected of having at least one neurodegenerative condition.

In certain embodiments, a subject to be treated by any of the methods disclosed herein can be a human having or suspected of having Alzheimer's disease. In some embodiments, a subject to be treated by any of the methods disclosed herein can be diagnosed with Alzheimer's disease via routine medical examination, e.g., laboratory tests, behavioral tests, computerized tomography (CT) scans, electroencephalogram, positron emission tomography (PET), and/or magnetic resonance imaging (MRI). In some embodiments, a subject to be treated by any of the methods disclosed herein can be a human patient having been diagnosed with mild Alzheimer's disease, moderate Alzheimer's disease, or severe Alzheimer's disease. Symptoms that can be associated with mild Alzheimer's disease can include memory loss of recent events, difficulty with problem-solving, complex tasks and sound judgments, changes in personality, difficulty organizing and expressing thoughts, and/or getting lost or misplacing belongings. Symptoms that can be associated with moderate Alzheimer's disease can include displays of increasingly poor judgment and deepening confusion, wandering behavior and possibly where the subject is in search of surroundings that feel more familiar, even greater memory loss, needing help with some daily activities, and/or significant changes in personality and behavior. Symptoms that can be associated with severe Alzheimer's disease can include loss of the ability to communicate coherently, requiring daily assistance with personal care, and/or experiencing a decline in physical abilities.

In other embodiments, a subject contemplated herein can be diagnosed with another neurodegenerative disorder where cognition in the subject is negatively affected. In accordance with these embodiments, a subject having such a condition can be diagnosed using standard methods and GM-CSF treatment regimens can commence when determined by a health professional. In certain embodiments, parameters disclosed herein can be used to compare to baseline data and used to optimize the GM-CSF treatment for the subject diagnosed with or having a neurodegenerative disorder.

In certain embodiments, effective concentrations of GM-CSF or a pharmaceutical composition disclosed herein can vary, as recognized by those skilled in the art, depending on route of administration, excipient usage, and co-usage with other active agents. For example, an “effective amount” of GM-CSF, such as sargramostim, can be the amount that alone, or together with further doses, produces the desired response, e.g., alleviating one or more symptoms associated with a neurodegenerative condition (e.g., decreased memory, decreased cognitive skill, declining physical abilities, increased inflammation). In some embodiments, such responses can be monitored by routine methods or can be monitored according to the methods disclosure herein. In some embodiments, the desired responses to treatment of a target neurodegenerative condition can also include delaying the onset or progress of neurodegenerative condition, e.g., a decline in physical abilities, cognitive state, and/or memory. In some embodiments, methods of monitoring one or more symptoms of the cognitive state in a human patient having a neurodegenerative condition can include, but are not limited to, a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), or a combination thereof.

In some embodiments, an effective concentration of GM-CSF or a pharmaceutical composition disclosed herein for use in a treatment regimen can be about 150 μg/m² to about 350 μg/m²/day. In some embodiments, an effective concentration of GM-CSF or a pharmaceutical composition disclosed herein for use can be about 200 μg/m²/day to about 300 μg/m²/day. In certain embodiments, an effective concentration of GM-CSF or a pharmaceutical composition disclosed herein for use in a subject can be about 250 μg/m²/day. In some embodiments, an effective concentration of sargramostim or similar GM-CSF disclosed herein for use in treating a subject can be about 150 μg/m² to about 350 μg/m²/day. In some embodiments, an effective concentration of sargramostim to treat a subject disclosed herein can be about 200 μg/m²/day to about 300 μg/m²/day. In some embodiments, an effective concentration of sargramostim for use in treating a subject can be about 250 μg/m²/day.

In certain embodiments, methods of the present disclosure can be used to personalize a GM-CSF dosing regimen recommendation for a subject having or suspected of having at least one neurodegenerative condition. In other embodiments, methods disclosed herein can include an initial GM-CSF dosing regimen or a GM-CSF pharmaceutical composition disclosed herein given to a subject for a first course of treatment of 5 days per week for a pre-determined period (e.g., about a week to about a month), which may be followed by one or more GM-CSF dosing regimen.

In some embodiments, an initial dosing regimen of about 100 μg/m²/day to about 400 μg/m²/day or about 200 μg/m²/day to about 300 μg/m²/day GM-CSF (e.g., sargramostim) can be given to a subject having or suspected of having at least one neurodegenerative condition for at least three to at least seven days a week (e.g., about 3 days, about 4 days, about 5 days, about 6 days, about 7 days). In some embodiments, an initial dose of about 100 μg/m²/day to about 400 μg/m²/day or about 200 μg/m²/day to about 300 μg/m²/day GM-CSF (e.g., sargramostim) can be given to a subject having or suspected of having at least one neurodegenerative condition for at least three to at least seven days a week for up to about one to about six weeks (e.g., about one week, about two weeks, about 3 weeks, about 4 weeks, about 5 weeks, about 6 weeks). In some embodiments, an initial dose of about 250 μg/m²/day GM-CSF (e.g., sargramostim) can be given to a subject having or suspected of having at least one neurodegenerative condition for one time for at least five days for up about least three weeks. In other embodiments, the concentration of GM-CSF and/or frequency of treatment can be altered depending on parameters assessed in the subject after a period of time.

In some embodiments, an initial dose of about 100 μg/m²/day to about 400 μg/m²/day or about 200 μg/m²/day to about 300 μg/m²/day GM-CSF (e.g., sargramostim) can be given to a subject having or suspected of having at least one neurodegenerative condition for at least three to at least seven days a week (e.g., about 3 days, about 4 days, about 5 days, about 6 days, about 7 days) for at least three to at least seven days a week for up to about one to six weeks (e.g., about one week, about two weeks, about 3 weeks, about 4 weeks, about 5 weeks, about 6 weeks). In some embodiments, an initial dose of about 100 μg/m²/day to about 400 μg/m²/day or about 200 μg/m² to about 300 μg/m²/day GM-CSF (e.g., sargramostim) can be given to a subject having or suspected of having at least one neurodegenerative condition for at least five days for up to at least three weeks. In some embodiments, an initial dose of about 250 μg/m²/day GM-CSF (e.g., sargramostim) can be given to a subject having or suspected of having at least one neurodegenerative condition for one time for at least five days for up about least three weeks.

In certain embodiments, at least one cognitive state exam can be performed prior to administration of an initial dose as disclosed herein for example, to establish a baseline cognitive measurement of a subject. In certain embodiments, at least one cognitive state exam can be performed after administration of an initial dose (e.g., after a dosing regimen up to about one to five weeks as disclosed herein) to establish a follow-up cognitive measurement of a subject. In some embodiments, the cognitive state exam performed before and/or after administration of an initial dose as disclosed herein can include, but is not limited to, a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), or a combination thereof.

In certain embodiments, at least one blood sample can be collected from a subject prior to administration of an initial GM-CSF dosing regimen as disclosed herein to establish a baseline blood sample. In certain embodiments, at least one blood sample can be collected from a subject after administration of the initial GM-CSF dosing regimen (e.g., after a dosing regimen up to about one to five weeks as disclosed herein) to establish a follow-up blood sample. In accordance with these embodiments, the at least one blood sample collected from the subject before and/or after administration of an initial GM-CSF dosing regimen can be analyzed as disclosed herein and compared in order to assess and administer a more individualized GM-CSF dosing regimen.

In some embodiments, a baseline blood sample and/or a follow-up blood sample can be used to determine white blood cells count. In other embodiments, a baseline blood sample and/or a follow-up blood sample can be used to determine an absolute number of at least one leukocyte. In certain embodiments, an absolute number of at least one leukocyte in a baseline blood sample and/or a follow-up blood sample can include an absolute number of monocytes, an absolute number of lymphocytes, and/or an absolute number of neutrophils. In some embodiments, a baseline blood sample and/or a follow-up blood sample can be used to determine the concentration of at least one inflammatory molecule (e.g. cytokine). In other embodiments, the concentration of at least one inflammatory molecule can include but is not limited to, a cytokine in a baseline blood sample and/or a follow-up blood sample. In accordance with these embodiments, at least one inflammatory molecule can include, but is not limited to interleukin (IL)-2, IL-6, IL-8, IL-10, Tumor Necrosis Factor (TNF)-alpha, or other proinflammatory cytokine or a combination thereof. In certain embodiments, a baseline blood sample and/or a follow-up blood sample can be used to determine the concentration of at least one neurodegenerative disorder biomarker (e.g. Alzheimer pathology biomarker). In other embodiments, the concentration of at least one neurodegenerative disorder biomarker (e.g. Alzheimer pathology biomarker) in a baseline blood sample and/or a follow-up blood sample can include Amyloid beta, Tau, ubiquitin C-terminal hydrolase L1 (UCH-L1), or a combination thereof. In some embodiments, a baseline blood sample and/or a follow-up blood sample can be used to determine ratio of albumin to globulin in the blood. In some embodiments, a baseline blood sample can be collected from a subject before administration of a dose (e.g., dosing regimen) of GM-CSF (e.g., sargramostim). In some embodiments, a baseline blood sample can be collected from a subject before administration of a first dose (i.e., an initial dose) of GM-CSF (e.g., sargramostim). In some embodiments, a baseline blood sample can be collected from a subject at about 48 hours to about less than 1 hour before administration of a dose (e.g., dosing regimen) of GM-CSF (e.g., sargramostim). In some embodiments, a baseline blood sample can be collected from a subject who has yet to be administered a dose (e.g., dosing regimen) of GM-CSF (e.g., sargramostim). In some embodiments, a follow-up blood sample can be collected from a subject after administration of a dose (e.g., dosing regimen) of GM-CSF (e.g., sargramostim). In some embodiments, a baseline blood sample can be collected from a subject at about less than 24 hours after administration of a dose (e.g., dosing regimen) of GM-CSF (e.g., sargramostim). In some embodiments, a baseline blood sample can be collected from a subject at about 1 day to about 4 months after administration of a dose (e.g., dosing regimen) of GM-CSF (e.g., sargramostim). In some embodiments, a follow-up blood sample can be collected from a subject after administration of a dose (e.g., dosing regimen) of GM-CSF (e.g., sargramostim) has been stopped. In some embodiments, a follow-up blood sample can be collected from a subject after administration of a dose of GM-CSF (e.g., sargramostim) has not been administered to the subject for at least about 24 hours. In some embodiments, a follow-up blood sample can be collected from a subject after administration of a dose of GM-CSF (e.g., sargramostim) has not been administered to the subject for at about 1 day to about 1 year, about 3 days to about 6 months, or about 1 week to about 3 months. In some embodiments, a follow-up blood sample can be collected from a subject after administration of a dose (e.g., dosing regimen) of GM-CSF (e.g., sargramostim) has been stopped and prior to administration of an additional dose (e.g., dosing regimen) of GM-CSF (e.g., sargramostim).

In certain embodiments, administration of an additional dose (e.g., dosing regimen) of GM-CSF (e.g., sargramostim) to a subject can be modified if, when comparing the baseline cognitive measurement to the follow-up cognitive measurement, there is a change in cognitive state. In certain embodiments, administration of an additional dose (e.g., dosing regimen) of GM-CSF (e.g., sargramostim) to a subject herein can stopped if, when comparing the baseline cognitive measurement to the follow-up cognitive measurement, there is a decline in cognitive state. In certain embodiments, administration of an additional dose (e.g., dosing regimen) of GM-CSF (e.g., sargramostim) to a subject herein can stopped for at least about 30 to about 60 days (e.g., about 30, 35, 40, 45, 50, 55, 60 days) and continued for at least about one to about seven days per week for up to about one to about six weeks if, when comparing the baseline cognitive measurement to the follow-up cognitive measurement, there is a decline in cognitive state. In certain embodiments, administration of an additional dose (e.g., dosing regimen) of GM-CSF (e.g., sargramostim) to a subject herein can be stopped for at least about 45 days and continued for at least about five days per week for up to about three weeks if, when comparing the baseline cognitive measurement to the follow-up cognitive measurement, there is a decline in cognitive state. In certain embodiments, administration of an additional dose (e.g., dosing regimen) of GM-CSF (e.g., sargramostim) to a subject herein can be given if, when comparing the baseline cognitive measurement to the follow-up cognitive measurement, there is a steady cognitive state or an increase in cognitive state.

In certain embodiments, administration of an additional dose (e.g., dosing regimen) of GM-CSF (e.g., sargramostim) to a subject can be modified if, when comparing the baseline blood sample to the follow-up blood sample, the absolute number of at least one leukocyte (e.g., absolute number of monocytes, absolute number of lymphocytes, absolute number of neutrophils) is changed. In certain embodiments, administration of an additional dose (e.g., dosing regimen) of GM-CSF (e.g., sargramostim) is not given to a subject herein if, when comparing the baseline blood sample to the follow-up blood sample, the absolute number of at least one leukocyte (e.g., absolute number of monocytes, absolute number of lymphocytes, absolute number of neutrophils) is increased. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks if, when comparing the baseline blood sample to the follow-up blood sample, the absolute number of at least one leukocyte (e.g., absolute number of monocytes, absolute number of lymphocytes, absolute number of neutrophils) is increased to a pre-determined level. In certain embodiments, administration of an additional dose (e.g., dosing regimen) of GM-CSF (e.g., sargramostim) to a subject herein can be stopped for at least about 45 days and later continued for at least about five days per week for up to about three weeks if, when comparing the baseline blood sample to the follow-up blood sample, the absolute number of at least one leukocyte (e.g., absolute number of monocytes, absolute number of lymphocytes, absolute number of neutrophils) is increased.

In certain embodiments, administration of an additional dose (e.g., dosing regimen) of GM-CSF (e.g., sargramostim) is not given to a subject herein if, when comparing the baseline blood sample to the follow-up blood sample, the absolute number of monocytes is increased by about 10% to about 50% or more (up to 100%); about 10% to about 45%; about 15% to about 40%; about 20% to about 35%; or about 25% to about 30% over baseline concentrations. In certain embodiments, administration of an additional dose (e.g., dosing regimen) of GM-CSF (e.g., sargramostim) is not given to a subject herein if, when comparing the baseline blood sample to the follow-up blood sample, the absolute number of monocytes is increased by about 30% over baseline concentrations. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks if, when comparing the baseline blood sample to the follow-up blood sample, the absolute number of monocytes is increased by about 10% to about 50% or more (up to 100%) (e.g., about 10%, 15%, 20%, 25%, 30%, 35% 40%, 45%, 50%, or 99%) over baseline concentrations. In certain embodiments, administration of an additional dose (e.g., dosing regimen) of GM-CSF (e.g., sargramostim) to a subject herein can be stopped for at least about 45 days and continued for at least about five days per week for up to about three weeks if when comparing the baseline blood sample to the follow-up blood sample, the absolute number of monocytes is increased by about 30% over baseline concentrations. In certain embodiments, administration of an additional dosing regimen of GM-CSF (e.g., sargramostim) to a subject herein can be given if when comparing the baseline blood sample to the follow-up blood sample, the absolute number of monocytes is increased by less than about 10% over baseline concentrations. In some embodiments, dosing regimens can be a single regimen, two, three or more dosing regimens depending on the parameters in the subject.

In certain embodiments, administration of an additional dose (e.g., dosing regimen) of GM-CSF (e.g., sargramostim) is not given to a subject herein if when comparing the baseline blood sample to the follow-up blood sample, the absolute number of lymphocytes is increased by about 10% to about 30% or more, up to 100% (e.g., about 10%, 15%, 20%, 25%, 30%, or 99%) over baseline concentrations. In certain embodiments, administration of an additional dose (e.g., dosing regimen) of GM-CSF (e.g., sargramostim) is not given to a subject herein if when comparing the baseline blood sample to the follow-up blood sample, the absolute number of lymphocytes is increased by about 15% over baseline concentrations. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks if when comparing the baseline blood sample to the follow-up blood sample, the absolute number of lymphocytes is increased by about 10% to about 30% or more, up to 100% (e.g., about 10%, 15%, 20%, 25%, 30%, or 99%) over baseline concentrations. In certain embodiments, administration of an additional dose (e.g., dosing regimen) of GM-CSF (e.g., sargramostim) to a subject herein can be stopped for at least about 45 days and continued for at least about five days per week for up to about three weeks if when comparing the baseline blood sample to the follow-up blood sample, the absolute number of lymphocytes is increased by about 15% over baseline concentrations. In certain embodiments, administration of an additional dose (e.g., dosing regimen) of GM-CSF (e.g., sargramostim) to a subject herein can be given if when comparing the baseline blood sample to the follow-up blood sample, the absolute number of lymphocytes is increased by less than about 10% over baseline concentrations.

In certain embodiments, administration of an additional dosing regimen of GM-CSF (e.g., sargramostim) to a subject herein can be modified if when comparing the baseline blood sample to the follow-up blood sample, the concentration of at least one inflammatory cytokine (e.g., IL-2, IL-6, IL-8, IL-10, Tumor Necrosis Factor (TNF)-alpha) is changed. In certain embodiments, administration of an additional dose (e.g., dosing regimen) of GM-CSF (e.g., sargramostim) is not given to a subject when comparing the baseline blood sample to the follow-up blood sample, the concentration of at least one inflammatory cytokine (e.g., IL-2, IL-6, IL-10, Tumor Necrosis Factor (TNF)-alpha) is increased. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks when comparing the baseline blood sample to the follow-up blood sample, the concentration of at least one inflammatory cytokine (e.g., IL-2, IL-6, IL-10, Tumor Necrosis Factor (TNF)-alpha) is increased. In certain embodiments, administration of an additional dose (e.g., dosing regimen) of GM-CSF (e.g., sargramostim) to a subject can be stopped for at least about 45 days and continued for at least about five days per week for up to about three weeks when comparing the baseline blood sample to the follow-up blood sample, the concentration of at least one inflammatory cytokine (e.g., IL-2, IL-6, IL-10, Tumor Necrosis Factor (TNF)-alpha) is increased. In certain embodiments, administration of an additional dose (e.g., dosing regimen) of GM-CSF (e.g., sargramostim) to a subject herein can be given if when comparing the baseline blood sample to the follow-up blood sample, the concentration of at least one inflammatory cytokine (e.g., IL-2, IL-6, IL-10, Tumor Necrosis Factor (TNF)-alpha) is less than about 10% over baseline concentrations, equal to baseline concentrations, or less than about 10% under baseline concentrations.

In certain embodiments, administration of an additional dose (e.g., dosing regimen) of GM-CSF (e.g., sargramostim) is not given to a subject herein if when comparing the baseline blood sample to the follow-up blood sample, the concentration of at least one inflammatory cytokine (e.g., IL-8) is decreased. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks (or five weeks) if when comparing the baseline blood sample to the follow-up blood sample, the concentration of at IL-8 is decreased. In certain embodiments, administration of an additional dose (e.g., dosing regimen) of GM-CSF (e.g., sargramostim) to a subject herein can be stopped for at least about 45 days and continued for at least about five days per week for up to about three weeks if when comparing the baseline blood sample to the follow-up blood sample, the concentration of IL-8 is decreased. In certain embodiments, administration of an additional dose (e.g., dosing regimen) of GM-CSF (e.g., sargramostim) to a subject herein can be given if when comparing the baseline blood sample to the follow-up blood sample, the concentration of at least one inflammatory cytokine IL-8 is less than about 10% over baseline concentrations, equal to baseline concentrations, or less than about 10% under baseline concentrations.

In certain embodiments, administration of an additional dose (e.g., dosing regimen) of GM-CSF (e.g., sargramostim) to a subject can be modified if when comparing the baseline blood sample to the follow-up blood sample, the concentration of at least one neurodegenerative condition-linked biomarker (e.g., Amyloid beta, Tau, UCH-L1), is changed. In certain embodiments, administration of an additional dose (e.g., dosing regimen) of GM-CSF (e.g., sargramostim) is not given to a subject if when comparing the baseline blood sample to the follow-up blood sample, the concentration of at least one neurodegenerative condition-linked biomarker (e.g., Amyloid beta, Tau, UCH-L1) is increased. In certain embodiments, administration of an additional dose (e.g., dosing regimen) of GM-CSF (e.g., sargramostim) to a subject can be stopped for at least about 30 to about 60 days (e.g., about 30, 35, 40, 45, 50, 55, 60 days) and continued for at least about one to about seven days per week for up to about one to about six weeks if when comparing the baseline blood sample to the follow-up blood sample, the concentration of at least one neurodegenerative condition-linked biomarker (e.g., Amyloid beta, Tau, UCH-L1) is increased. In certain embodiments, administration of an additional dose (e.g., dosing regimen) of GM-CSF (e.g., sargramostim) to a subject can be stopped for at least about 45 days and continued for at least about five days per week for up to about three weeks if when comparing the baseline blood sample to the follow-up blood sample, the concentration of at least one neurodegenerative condition-linked biomarker (e.g., Amyloid beta, Tau, UCH-L1) is increased. In certain embodiments, an increase in at least one neurodegenerative condition-linked biomarker contemplated herein can be about a 10% increase or more.

In certain embodiments, administration of an additional dosing regimen of GM-CSF (e.g., sargramostim) is not given to a subject herein if when comparing the baseline blood sample to the follow-up blood sample, the concentration of Tau is increased to a pre-determined level. In certain embodiments, administration of an additional dosing regimen of GM-CSF (e.g., sargramostim) is not given to a subject herein if when comparing the baseline blood sample to the follow-up blood sample, the concentration of Tau is increased to over about 20% to about 40% compared to a control subject not having a neurodegenerative condition. In certain embodiments, administration of an additional dosing regimen of GM-CSF (e.g., sargramostim) is not given to a subject herein if when comparing the baseline blood sample to the follow-up blood sample, the concentration of Tau is increased to about 30% compared to baseline levels or a control subject no having a neurodegenerative condition. In certain embodiments, administration of an additional dosing regimen of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks if when comparing the baseline blood sample to the follow-up blood sample, the concentration of Tau is increased by about 30%. In other embodiments, complete clinical and cognitive assessment can be performed and all GM-CSF dosing regimens ceased. In certain embodiments, administration of an additional dosing regimen of GM-CSF (e.g., sargramostim) to a subject herein can be stopped for at least about 45 days and continued for at least about five days per week for up to about three weeks if when comparing the baseline blood sample to the follow-up blood sample, the concentration of Tau is increased by about 30%. In certain embodiments, administration of a dosing regimen of GM-CSF (e.g., sargramostim) can be given to a subject herein if when comparing the baseline blood sample to the follow-up blood sample, the concentration of Tau is increased by less than about 10%, or less than about 5% or the same concentration.

In certain embodiments, administration of an additional dosing regimen of GM-CSF (e.g., sargramostim) is not given to a subject herein if when comparing the baseline blood sample to the follow-up blood sample, the concentration of UCH-L1 is increased to a pre-determined level. In certain embodiments, administration of an additional dosing regimen of GM-CSF (e.g., sargramostim) is not given to a subject herein if when comparing the baseline blood sample to the follow-up blood sample, the concentration of UCH-L1 is increased by about 20% to about 40% (e.g. about 20%, 25%, 30%, 35%, 40%). In certain embodiments, administration of an additional dose (e.g., dosing regimen) of GM-CSF (e.g., sargramostim) is not given to a subject herein if when comparing the baseline blood sample to the follow-up blood sample, the concentration of UCH-L1 is increased by about 30%. In certain embodiments, administration of an additional dosing regimen of GM-CSF (e.g., sargramostim) to a subject herein can be stopped for at least about 45 days and continued for at least about five days per week for up to about three weeks if when comparing the baseline blood sample to the follow-up blood sample, the concentration of UCH-L1 is increased by about 30%. In certain embodiments, administration of an additional dosing regimen of GM-CSF (e.g., sargramostim) can be given to a subject herein if when comparing the baseline blood sample to the follow-up blood sample, the concentration of UCH-L1 is increased by less than about 10% or less than about 5% or the same concentration.

In certain embodiments, administration of an additional dose (e.g., dosing regimen) of GM-CSF (e.g., sargramostim) is not given to a subject herein if, when comparing the baseline blood sample to the follow-up blood sample, the ratio of albumin to globulin in the blood is below the baseline ratio of albumin to globulin by at least about 9%. In accordance with these embodiments, administration of an additional dose of GM-CSF is not given to a subject herein if, when comparing the baseline blood sample to the follow-up blood sample, the ratio of albumin to globulin in the blood is about 9% below, about 10% below, about 11% below, about 12% below, about 13%, or more below the baseline ratio of albumin to globulin.

In certain embodiments, administration of an additional dose (e.g., dosing regimen) of GM-CSF (e.g., sargramostim) to a subject herein can be stopped and continued if, when comparing the baseline blood sample to the follow-up blood sample, the ratio of albumin to globulin in the blood is below the baseline by at least 5% or less. In accordance with these embodiments, administration of an additional dose (e.g., dosing regimen) of GM-CSF (e.g., sargramostim) to a subject herein can be stopped and continued (i.e., restarted) if, when comparing the baseline blood sample to the follow-up blood sample, the ratio of albumin to globulin in the blood is about 5% below, about 4% below, about 3% below, about 2% below, about 1% below, or about equal to the baseline ratio of albumin to globulin. In certain embodiments, administration of an additional dose (e.g., dosing regimen) of GM-CSF (e.g., sargramostim) to a subject herein can be stopped for at least about 30 to about 60 days (e.g., about 30, 35, 40, 45, 50, 55, 60 days) and continued for at least about one to about seven days per week for up to about one to about six weeks if, when comparing the baseline blood sample to the follow-up blood sample, the ratio of albumin to globulin in the blood is below the baseline by at least 5% or less. In certain embodiments, administration of an additional dose (e.g., dosing regimen) of GM-CSF (e.g., sargramostim) to a subject herein can be stopped for at least about 45 days and continued for at least about five days per week for up to about three weeks if when comparing the baseline blood sample to the follow-up blood sample, the ratio of albumin to globulin is below the baseline by at least 5% or less.

In some embodiments, the subject to be treated by the methods described herein can be a human subject or patient who has undergone or is currently undergoing at least one anti-neurodegenerative therapy. In other embodiments, the anti-neurodegenerative therapy can be complete. In some embodiments, the anti-neurodegenerative therapy can be ongoing. In other embodiments, the subject can be undergoing a combined therapy involving a GM-CSF (e.g., sargramostim) dosing regimen disclosed herein and at least a second anti-neurodegenerative therapy. In accordance with these embodiments, an exemplary anti-neurodegenerative therapy can include, but is not limited to, dopaminergic treatments, cholinesterase inhibitors, antipsychotic drugs, analgesic drugs, anti-inflammatories, use of deep brain stimulation, and the like.

In certain embodiments, the present disclosure includes kits for use in methods described herein. A kit for therapeutic use as described herein can include one or more containers having GM-CSF (e.g., sargramostim). In some embodiments, the kit can additionally have instructions for use of GM-CSF (e.g., sargramostim) or compositions thereof in any of the methods described herein. The instructions can include a description of administration of GM-CSF (e.g., sargramostim) or a pharmaceutical composition to be administered to a subject to achieve the intended result or outcome in a subject. The kit can further have a description of selecting a subject suitable for treatment based on identifying whether the subject is in need of the treatment. In some embodiments, the instructions include a description for administering a GM-CSF (e.g., sargramostim) dosing regimen or a pharmaceutical composition disclosed herein to a subject who has or is suspected of having at least one neurodegenerative condition or disease.

In some embodiments, kits can have instructions relating to the use of GM-CSF (e.g., sargramostim) or the pharmaceutical composition according to the methods disclosed herein. In some embodiments, instructions within the kit can generally include information as to dosage, dosing schedule, and route of administration for the intended treatment. In some embodiments, instructions of kits herein can include a description of optimizing the dose of GM-CSF (e.g., sargramostim) in a subject having neurodegenerative condition using changes in cognitive state as a biomarker. In some embodiments, instructions of kits herein can include a description of performing at least one cognitive state exam (e.g., comprises a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL)).

In some embodiments, instructions of kits can include a description of optimizing the dose of GM-CSF (e.g., sargramostim) in a subject having neurodegenerative condition using changes in blood biomarkers (e.g., absolute number of at least one leukocyte, a concentration of at least one inflammatory cytokine, a concentration of at least one Alzheimer brain pathology biomarker). In some embodiments, kits can include containers (e.g., tubes, vials, buffers, syringes, and the like) to collect blood and/or syringes for drawing blood from a subject for testing or storage for later analysis.

In some embodiments, the kit can have one or more containers. In some embodiments, containers can be unit doses, bulk packages (e.g., multi-dose packages) or sub-unit doses. In some embodiments, instructions supplied in kits of the disclosure can be written instructions on a label or package insert. In some embodiments, the label or package insert can indicate that the pharmaceutical compositions are used for treating, delaying the onset, and/or alleviating a disease or disorder in a subject.

In certain embodiments, kits provided herein are in suitable packaging. Suitable packaging includes, but is not limited to, vials, bottles, jars, flexible packaging, and the like. In some embodiments, kits can have packages for use in combination with a specific device, such as an inhaler, nasal administration device, or an infusion device. In some embodiments, a kit may have a sterile access port (for example, the container may be an intravenous solution bag or a vial having a stopper pierceable by a hypodermic injection needle). In some embodiments, a container may also have a sterile access port.

In certain embodiments, kits herein can provide additional components such as buffers and interpretive information. In some embodiments, a kit herein can have a container and a label or package insert(s) on or associated with the container. In some embodiments, the disclosure provides articles of manufacture comprising contents of the kits described above.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

In one exemplary method, a randomized, double-blind, placebo-controlled phase II trial was performed to test the safety and efficacy of sargramostim treatment in mild-to-moderate Alzheimer's disease (AD) participants, defined by clinical diagnosis and a screening Mini-Mental State Exam (MMSE) score of 10-26 inclusive. Inclusion criteria for the study were as follows:
1. age 55 to 85 years;
2. should have a mild-to-moderate AD diagnosis (MMSE 10-26 inclusive);
3. should have evidence of elevated cortical amyloid by PET using florbetapir F18 (Amyvid) [i.e. a positive scan], assessed qualitatively according to the Amyvid product label.
4. if on anti-dementia treatment should be on stable treatment for at least 2 months (i.e. cholinesterase inhibitor and/or Memantine or Axona);
5. stable on all other medications for at least 30 days prior to screen;
6. should be fluent in English;
7. should be physically able to participate by medical history, clinical exam and tests;
8. should have a study partner to accompany them to scheduled visits.
   Exclusion criteria for the study were as follows:
1. clinically relevant arrhythmias;
2. a resting pulse less than 50;
3. active cancer other than non-melanoma skin cancers;
4. use of another investigatory drug within 2 months of screening;
5. significant stroke or head trauma by history or Mill;
6. contraindication for having a Mill;
7. diagnostic and Statistical Manual of Mental Disorders-IV criteria for a current major psychiatric disorder;
8. sensitivity to yeast or yeast products;
9. impaired kidney function as measured by a Glomerular Filtration Rate less than 60 milliliters/min;
10. preexisting fluid retention, pulmonary infiltrates, or congestive heart failure;
11. history of moderate-to-severe lung disease;
12. history of moderate-to-severe liver disease;
13. pregnant women, or any women who feel they are likely to become pregnant during the study;
14. prisoners.

Participants who met eligibility criteria were randomized to receive either sargramostim (20 participants, standard FDA dosage 250 mcg/m²/day subcutaneous injection [SC], five days/week for three weeks) or placebo (20 participants, SC saline, five days/week for three weeks).

At the baseline visit, each participant underwent neurological and cognitive assessments, safety and biomarker blood draws, and an MRI scan before being administered their first dose of sargramostim or placebo. Participants returned daily, five days/week for three weeks, for SC sargramostim or placebo and for follow-up visits at 45 days (FU1) and 90 days (FU2) after the end of treatment (EOT). Neuropsychological tests, MMSE, ADAS-Cog memory subscale (ADAS delayed word recall+ADAS word recognition+ADAS orientation+ADAS word recall avg), ADAS-Cog13, Alzheimer's Disease Cooperative Study Activities of Daily Living Scale (ADCS-ADL), Clinical Dementia Rating scale Sum of Boxes (SDR-SB), and Trail Making Test A (TRAILS-A) were performed at baseline, EOT, FU1, and FU2. Safety/biomarker blood draws, MRI, and, for the last half of the study, amyloid-PET images were collected. Study data were managed using REDCap electronic data capture tools.

Cognitive tests were performed as follows. Education corrected Folstein Mini-Mental State Exam MMSE is a standard cognitive assessment used by all ADRCs including the FADRC to identify and monitor AD subjects and was performed according to the standard procedures. ADAS was designed to measure the severity of the most important symptoms of AD. Its subscale ADAS-cog is the most popular cognitive testing instrument used in clinical trials of nootropics (drugs or agents that improve cognitive function). The ADAS-cog (Alzheimer's Disease Assessment Scale-cognitive subscale) consisted of 11 tasks that measured the disturbances of memory, language, praxis, attention and other cognitive abilities which are often referred to as the core symptoms of AD. ADCS/ADL (Alzheimer's Disease Cooperative Study Activities of Daily Living Inventory) was administered as a caregiver rated questionnaire of 23 items, with possible scores over a range of 0-78, where 78 implies full functioning with no impairment. The ADCS-ADL assessed functional capacity across a wide spectrum of severity and will be the primary tool for collecting ADL data. Psychomotor speed was assessed by the Trail Making Test-A (TRAILS A), a timed test in which subjects connected a series of numbers randomly placed on a page. Executive function was assessed using Trail Making Test-B (TRAILS B), a sensitive test of cognitive flexibility and psychomotor speed in which subjects connected a series of alternating numbers and letters placed randomly on a page. Psychomotor speed and cognitive vigilance was assessed using Mohs Cancellation Task, a timed task involving cancellation of target stimuli in a larger array of distractors. CDR (Clinical Dementia Rating) was administered as a caregiver and subject based interview to assess changes in domains such as memory, orientation, judgment and problem solving, community affairs, home and hobbies, and personal care. Each domain was rated as 0 (no dementia), 0.5 (uncertain dementia), 1 (mild dementia), 2 (moderate dementia), or 3 (severe dementia). All raters in this trial completed Alzheimer's Disease Research Center CDR Training.

Blood biomarkers were collected and assessed as follows. CBC with differential biomarkers were collected twice weekly from baseline visit to end-of-treatment and at 45 day and 90 day follow-up visits for each participant, whole blood was collected in an EDTA vacutainer and sent to a contracted laboratory for analyses of complete blood counts (CBC) with differential. Results were sent to the unblinded trial biostatistician to determine sargramostim or placebo group effects on leukocyte populations (i.e., neutrophil, monocyte, and lymphocyte) and albumin/globulin ratios at each visit time point. For plasma biomarkers, after collection of whole blood in EDTA vacutainers, each vacutainer sample was centrifuged at 1,500×g for 15 min at 22° C. and plasma removed within two hours of collection. Plasma was then centrifuged again at 2,200×g for 10 min at 4° C. to pellet any remaining cells and debris, and then plasma was transferred into 250 μL aliquots and immediately stored at −80° C. All EDTA blood vacutainer tubes and sample aliquots were labeled only with the participant's unique trial ID, the date sample was obtained, and the participant trial visit (i.e., baseline, end-of-treatment, 45-day follow-up, or 90-day follow-up), and all laboratory technicians processing the blood or performing biomarker assays remained blinded to any other identifying participant information or treatment group (i.e., sargramostim or placebo). On days of biomarker analyses, frozen samples were selected in the sample management database, removed from the −80° C. freezer, thawed on ice, and processed according to manufacturer instructions for either MSD (Meso Scale Diagnostics, Inc.) or SIMOA® (Quanterix) analyses. For each participant, their baseline, end-of-treatment, 45-day follow-up, and 90-day follow-up visit samples were analyzed together on the same assay plate, and each visit's sample was analyzed in triplicate. For MSD analyses, selected frozen plasma samples were transferred on dry ice to the CU Human Immune Monitoring Shared Resource (HIMSR) core facilities for thawing, processing, and running the V-PLEX Proinflammatory Panel 1 Human Kit assay, which measures the IFN-γ, IL-1β, IL-2, IL-4, IL-6, IL-8, IL-10, IL-12p70, IL-13, and TNF-α analytes in pg/ml. In preparation for SIMOA analyses using the Neuro 3-Plex A (Aβ40, Aβ42, Tau) and Neuro 4-Plex B (Nf-Light®, Tau, GFAP, UCH-L1) assays, selected frozen plasma samples were thawed on ice and transported to the CU Neurology Translational Core for analyses using the Quanterix SR-X™ Ultra-Sensitive Biomarker Detection System with SIMOA® bead technology to measure analytes at fg/ml levels. All MSD and SIMOA results were sent to the trial's unblinded biostatistician for determining any biomarker changes at the end-of-treatment, 45 day and 90 day follow-up visits, as compared to baseline, and for any differences between the sargramostim and placebo groups at the end-of-treatment, 45 day and 90 day follow-up visit time points.

Amyloid-PET images were collected and assessed as follows. For the last half of the study, PET amyloid imaging was required as an inclusion criterion to assure that the participants indeed had amyloid positive Alzheimer's disease. Ten participants were assessed by florbetapir (i.e., Amyvid) PET imaging (six sargramostim and four placebo) at screening and at 45 days post treatment follow-up visits. When Amyvid was no longer available, two enrolled participants were determined to be amyloid positive by historical amyloid scan, and for eight participants PET imaging with Pittsburgh Compound B at screening and at the 45-day follow-up visit. All participants diagnosed as having mild-to-moderate AD by the screening physician and then assessed by amyloid PET were indeed amyloid positive. Amyloid PET images were analyzed by Bioclinica. Freesurfer (version 5.3) was used to obtain regions of interest segmentations on T1 MRI data. Florbetapir and PiB PET data were motion corrected by realignment to the first frame of the raw image file and averaged across frames, smoothed to achieve uniform resolution of 6.5 mm in plane and 7 mm axial FWHM, and registered to the MM data in T1 native space. The composite cortical Standard Uptake Value Ratio (SUVr) was computed using a grouping of four larger cortical regions including lateral temporal, frontal, parietal (with precuneus), and anterior/posterior cingulate, equally weighted, and used as a standard ADNI florbetapir processing method. The whole cerebellum was used as a reference region for SUVr. Centiloid conversion aligns amyloid PET measures across tracers. Bioclinica SUVr pipeline was converted to standard Centiloid scale using standard methods.

Brain MM was performed for each participant at baseline, end of treatment (15 days), and first follow-up (45 days after the end of treatment). MRI was performed without intravenous gadolinium and included 3D T1, axial T2, axial FLAIR, diffusion-weighted images (DWI), and susceptibility weighted images (SWI). Each MRI study was visually reviewed and formally interpreted by a board-certified neuroradiologist to ensure no structural or physiological abnormalities that would preclude enrollment and to assess the potential development of adverse changes in the brain following treatment (i.e., Amyloid Related Imaging Abnormalities [ARIAS]), such as vasogenic edema and microhemorrhage. The presence of microhemorrhage was assessed on SWI, an imaging technique highly sensitive to hemosiderin and other blood products. No significant changes in gross parenchymal volume, white matter disease, or baseline microhemorrhage was observed for any participant over the MRI scans. Quantitative morphometry of T1 images was performed for the purposes of PET analysis, as described in the above section.

Safety (Primary Endpoint). Data and Safety Monitoring Board (DSMB) assessments of study events found no drug-related serious adverse events (SAEs), including no amyloid-related imaging abnormalities (ARIAs, determined by visual reads for microhemorrhages or edema) (Table 1).

TABLE 1

| | Total | | |
|---|---|---|---|
| | Sargramo- | Total | |
| Body System and Preferred | stim | Placebo | Total |
| Term | N = n**** | N = n*** | N = n*(n %)** |
| Overall | 59 | 36 | 95 |
| Cardiovascular | 5 | 2 | 7 (15.9) |
| Dizziness | 1 | 0 | 1 (2.3) |
| Heart Murmur | 1 | 0 | 1 (2.3) |
| High Blood Pressure, recurrent | 0 | 1 | 1 (2.3) |
| Lightheaded, recurrent | 0 | 1 | 1 (2.3) |
| Low Blood Pressure w/dizziness | 1 | 0 | 1 (2.3) |
| Myocardial Infarction | 1 | 0 | 1 (2.3) |
| Pain, chest | 1 | 0 | 1 (2.3) |
| Constitutional | 6 | 5 | 11 |
| Chills | 1 | 1 | 2 (4.5) |
| Fatigue | 2 | 4 | 6 (13.9) |
| Flu-like viral syndrome | 1 | 0 | 1 (2.3) |
| Malaise | 1 | 0 | 1 (2.3) |
| Vaso-Vagal | 1 | 0 | 1 (2.3) |
| Dental | 1 | 0 | 1 |
| Dental Surgery | 1 | 0 | 1 (2.3) |
| Dermatological | 16 | 5 | 21 |
| Cellulitis | 1 | 0 | 1 (2.3) |
| Cold Sore | 2 | 0 | 2 (4.5) |
| Injection Site Reaction* | 9 | 5 | 14 (31.8) |
| Rash | 3 | 0 | 3 (6.8) |
| Traumatic Bruise | 1 | 0 | 1 (2.3) |
| ENT | 2 | 0 | 2 |
| Hoarseness | 1 | 0 | 1 (2.3) |
| Sinus Pain | 1 | 0 | 1 (2.3) |
| Gastrointestinal | 8 | 5 | 13 |
| Abnormal discomfort | 1 | 0 | 1 (2.3) |
| Constipation | 1 | 0 | 1 (2.3) |
| Diarrhea | 2 | 3 | 5 (11.4) |
| Nausea | 3 | 1 | 4 (9.1) |
| Vomiting | 1 | 1 | 2 (4.5) |
| Musculoskeletal | 8 | 11 | 19 |
| Degenerative Arthritis | 0 | 1 | 1 (2.3) |
| Dupuytren's Contracture | 1 | 0 | 1 (2.3) |
| Fall | 1 | 4 | 5 (11.4) |
| Foot Cramp | 1 | 0 | 1 (2.3) |
| Muscle Weakness | 1 | 0 | 1 (2.3) |
| Pain/Myalgia | 4 | 5 | 9 (20.4) |
| Spinal Stenosis | 0 | 1 | 1 (2.3) |
| Neurological | 9 | 2 | 11 |
| Headache, Benign NOS | 8 | 2 | 10 (22.7) |
| Small Vessel Infarct | 1 | 0 | 1 (2.3) |
| Psychological | 0 | 2 | 2 (4.5) |
| Anxiety/Panic Attack | 0 | 1 | 1 (2.3) |
| Depression | 0 | 1 | 1 (2.3) |
| Respiratory | 4 | 4 | 8 (18.2) |
| Pneumonia | 0 | 1 | 1 (2.3) |
| Short of Breath | 1 | 0 | 1 (2.3) |
| Upper Respiratory Infection | 3 | 3 | 6 (13.6) |

*Number of participants experiencing an adverse event (participant is to be counted only once for each adverse event)
**% of total number of participants in the study
***Number of participants experiencing an adverse event who were on placebo
****Number of participants experiencing an adverse event who were on sargramostim

The most common sargramostim-associated AEs were dermatological (16 for sargramostim vs. 5 for placebo), gastrointestinal (8 for sargramostim vs. 5 for placebo), and headache (8 for sargramostim vs. 2 for placebo).

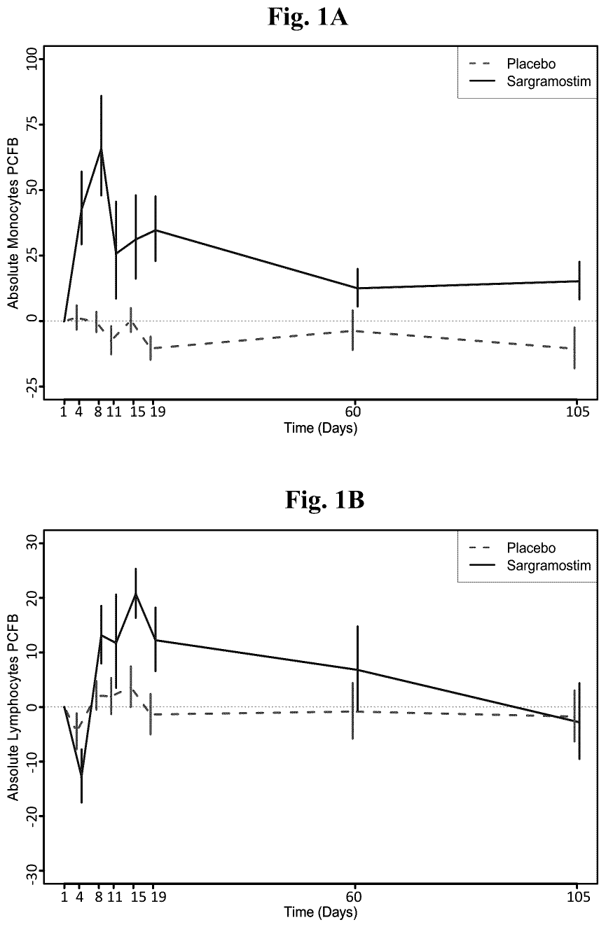

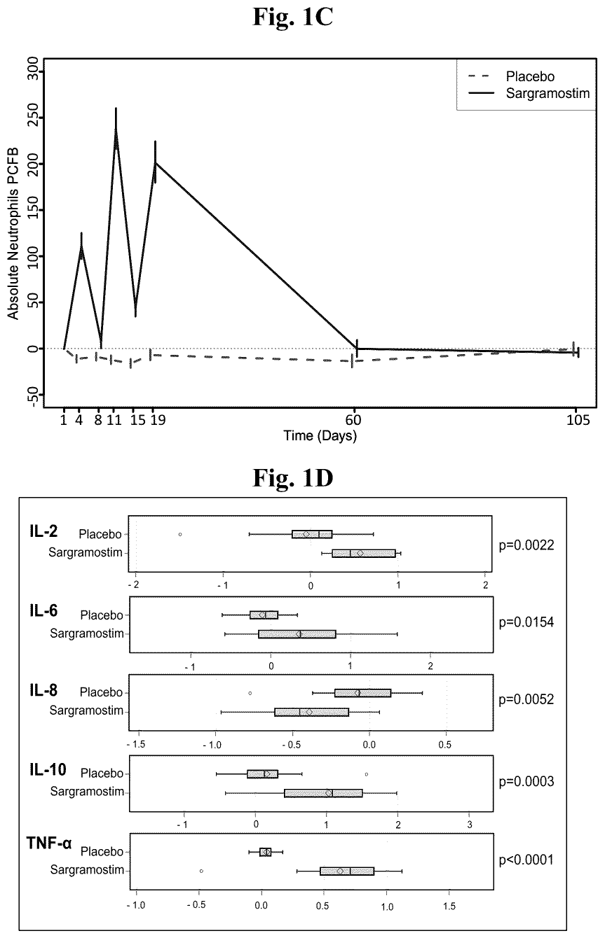

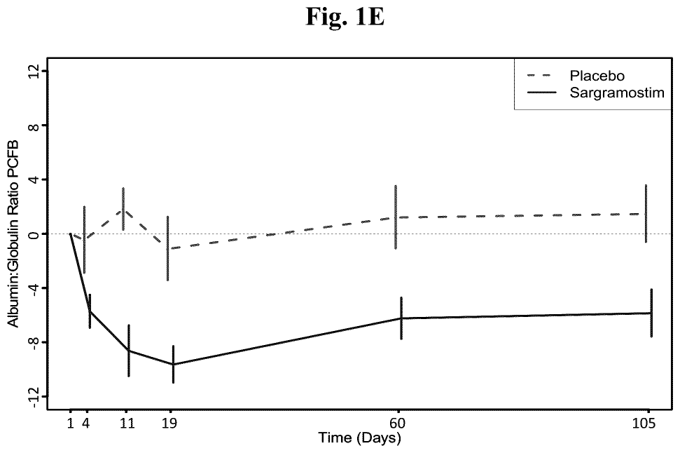

Innate Immune System Activation. To confirm an effect of GM-CSF/sargramostim on the innate immune system, were performed, which showed that monocytes (FIG. 1A), lymphocytes (FIG. 1B), and neutrophils (FIG. 1C) were all statistically significantly increased during the treatment phase (15 injections over three weeks) in the sargramostim group compared to the placebo group (p=0.0005, p=0.0512) (Table 2).

TABLE 2

| | | Absolute | | Absolute | | Absolute | |
|---|---|---|---|---|---|---|---|
| | | Monocytes | | Lymphocytes | | Neutrophils | |
| Treatment | Time | Mean | Std Dev | Mean | Std Dev | Mean | Std Dev |
| Placebo | Baseline (Day 1) | 534.40 | 176.05 | 1527.25 | 440.65 | 4767.30 | 1196.70 |
| | Day 4 | 542.85 | 177.84 | 1439.75 | 331.82 | 4222.00 | 1026.76 |
| | Day 8 | 549.57 | 186.86 | 1503.68 | 366.42 | 4276.11 | 872.71 |
| | Day 11 | 497.85 | 164.47 | 1539.85 | 364.012 | 4141.10 | 794.30 |
| | Day 15 | 535.95 | 183.74 | 1574.63 | 408.14 | 4022.37 | 828.55 |
| | Day 19 | 488.68 | 165.17 | 1501.74 | 439.85 | 4326.68 | 973.98 |
| | Follow Up 1 | 583.29 | 249.36 | 1482.29 | 383.65 | 4415.71 | 1418.97 |
| | Follow Up 2 | 545.40 | 223.53 | 1482.67 | 383.10 | 4616.73 | 998.51 |
| Sargranoslim | Baseline (Day 1) | 483.90 | 148.25 | 1650.30 | 443.623 | 3932.30 | 727.31 |
| | Day 4 | 668.84 | 201.38 | 1447.11 | 426.11 | 8322.84 | 1754.67 |
| | Day 8 | 811.42 | 301.37 | 1893.21 | 457.73 | 4398.74 | 1554.60 |
| | Day 11 | 658.65 | 322.70 | 1909.35 | 735.70 | 13439.4 [symbol] | 3127.31 |
| | Day 15 | 669.65 | 314.63 | 1990.35 | 523.50 | 5927.25 | 2194.59 |
| | Day 19 | 661.55 | 237.03 | 1871.20 | 574.28 | 12157.8 [symbol] | 3329.15 |
| | Follow Up 1 | 519.31 | 126.64 | 1757.77 | 545.44 | 3792.54 | 926.90 |
| | Follow Up 2 | 545.27 | 116.99 | 1761.45 | 601.96 | 3819.91 | 1202.58 |

[symbol]

indicates data missing or illegible when filed

The shorter half-life of neutrophils was revealed by the fact that when a weekend intervened after an injection of GM-CSF/sargramostim, the absolute neutrophil counts dropped, but then increased again during active treatment. The Meso-Scale Discovery system was used to assess changes in in plasma inflammatory cytokines with sargramostim treatment. At EOT compared to baseline, sargramostim treatment leads to statistically significant increases in IL-2 (p=0.0022), IL-6 (p=0.0154), IL-10 (p=0.0003), and TNF-alpha (p<0.0001), and a decrease in IL-8 (p=0.0052) (FIG. 1D). The ratio of plasma albumin to globulin was used to assess inflammation and its acute phase response and a statistically significant decrease in the albumin/globulin ratio was observed (FIG. 1E, Table 3), illustrating the immune-modulatory activities of GM-CSF.

TABLE 3

Plasma Albumin to
Globulin Ratio

| Treatment | Time | Mean | Std Dev |
|---|---|---|---|
| Placebo | Screening Visit | 1.59 | 0.30 |
| | Day 4 | 1.58 | 0.20 |
| | Day 11 | 1.62 | 0.24 |
| | Day 15 | 1.57 | 0.25 |
| | Follow Up 1 | 1.61 | 0.23 |
| | Follow Up 2 | 1.61 | 0.24 |
| Sargranoslim | Screening Visit | 1.66 | 0.22 |
| | Day 4 | 1.56 | 0.21 |
| | Day 11 | 1.52 | 0.23 |
| | Day 19 | 1.50 | 0.20 |
| | Follow Up 1 | 1.55 | 0.20 |
| | Follow Up 2 | 1.56 | 0.18 |

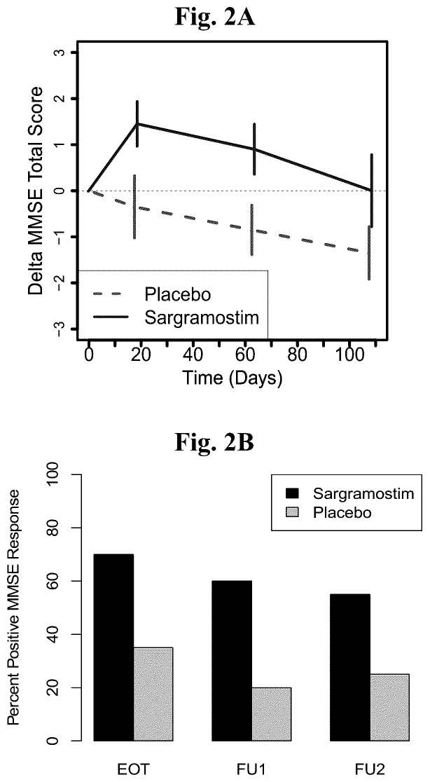

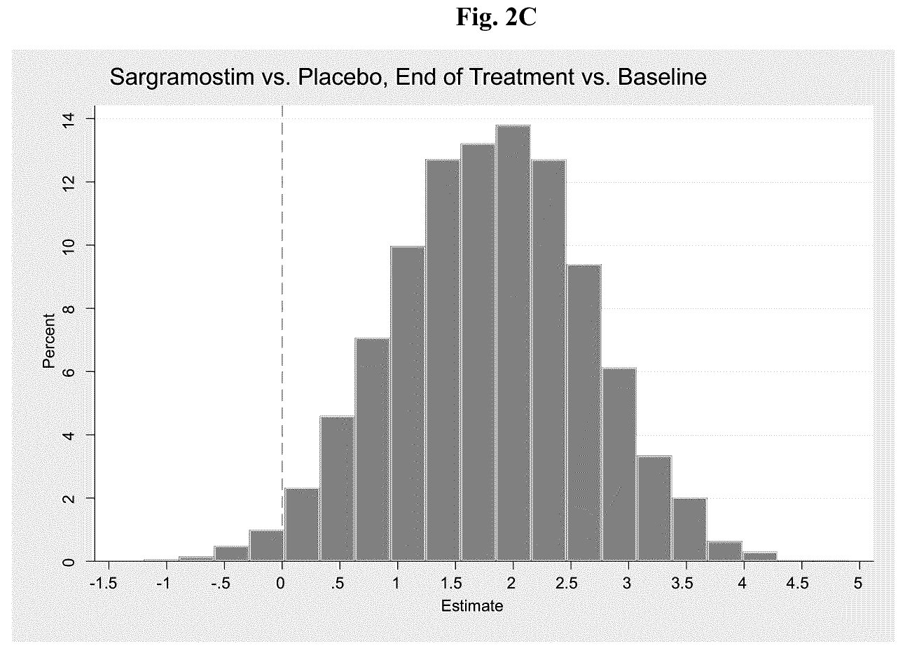

Efficacy (Secondary/Exploratory Endpoints). Analyses for potential efficacy revealed a statistically significant positive treatment effect of sargramostim on the Mini-Mental State Exam (MMSE), which was selected as an outcome measure based on its superior sensitivity to temporal changes. Specifically, at EOT, the mean MMSE total score change in the sargramostim group was 1.45 units higher relative to baseline (p=0.0074) (FIG. 2A; Table 4). The difference in mean change from baseline in MMSE total scores between the sargramostim and placebo groups was 1.80 (p=0.0370) at EOT and 1.75 (p=0.0272) at FU1 (45 days after EOT). The improvement in total MMSE score occurred in: (a) 70% of sargramostim-treated participants compared to 35% of placebo-treated participants at the EOT (p=0.0267), (b) 60% of sargramostim-treated participants compared to 20% of placebo-treated participants at FU1 (p=0.0098), and (c) 55% of sargramostim-treated participants compared to 25% placebo-treated participants at FU2 (trend; p=0.0528) (FIG. 2B). Combining the results to identify an overall treatment effect shows that 16 of 20 (80%) sargramostim-treated participants can be considered ‘responders’ in that they showed a higher MMSE score compared to baseline at either the EOT or at the 45-day follow-up visit, compared to only 7 of 20 (35%) placebo-treated participants (p=0.0040).

Study participants were randomized, and although the mean MMSE scores at screening were not significantly different between the sargramostim and placebo groups, they were at baseline (−3.65, 95% CI: [−6.71, −0.59], p=0.0207). Using baseline adjustment models, the treatment effect was statistically non-significant at the EOT (1.60, 95% CI: [−0.21, 3.40], p=0.0808) and was statistically significant at FU1 (1.80, 95% CI: [0.15, 3.46], p=0.0338).

To assess the effect of sargramostim treatment on the participants throughout the study, the 20 sargramostim-treated participants and 20 placebo-treated participants were each divided into 10,000 random subsamples of 10 participants each, and for each simulated data set, a model was run, generating estimates for the means and contrasts. For more than 98% of the simulated data sets, the estimate for change from baseline to the EOT within the sargramostim group, and the estimates for the treatment effects on change from baseline to end of treatment and change from baseline to FU1 were greater than zero. The mean Mini-Mental State Exam (MMSE) scores were calculated and graphed as a delta distribution. The distribution was approximately Gaussian and the mean and median delta was 1.8, as was the delta of the entire 20 sargramostim-treated participants and 20 placebo-treated participants analyzed in FIGS. 2A and 2B, showing that the statistically significant difference between the sargramostim-treated participants and the placebo-treated participants at the EOT reflected all parts of the trial (FIG. 2C). No significant difference between the mean total MMSE scores of participants enrolled during the first and second halves of the trial was found.

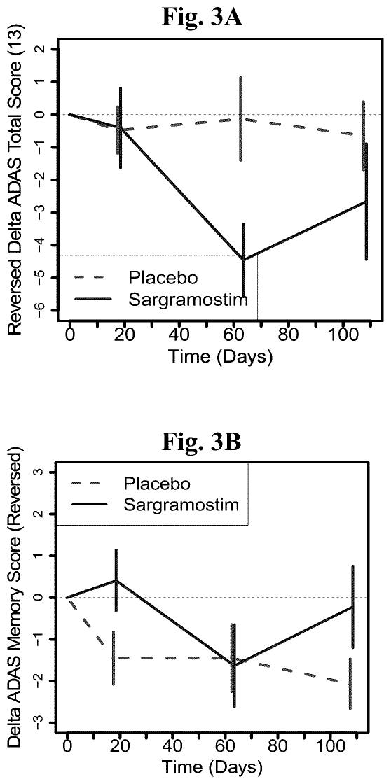

There were no significant changes in ADAS-Cog-13 scores between sargramostim-treated participants and placebo-treated participants during the treatment phase (through the EOT ˜day 19). At FU1, 45 days after the EOT, the ADAS-Cog-13 showed a statistically significant increase (worsening) in the sargramostim group compared to baseline (4.46, 95% CI: [2.11, 6.82], p=0.0009), and compared to the placebo group (4.33, 95% CI: [0.90, 7.76], p=0.0147) and then returned to the level of the placebo group at the 90-day follow-up visit. The treatment effect was stronger when baseline ADAS was adjusted for (5.54, 95% CI: [2.31, 8.78], p=0.0013), and there was a statistically significant effect of baseline on expected change score at the 45-day follow-up (−0.173 per scale unit, 95% CI: [−0.298, −0.049], p=0.0077). (FIG. 3A; Table 4).

To compare to the memory-predominant MMSE measure, the memory domain subscale of ADAS-Cog-13 (ADAS delayed word recall+ADAS word recognition+ADAS orientation+ADAS word recall avg) was analyzed. At the EOT, which is when the MMSE showed a statistically significant improvement (See FIGS. 2A-2C), the ADAS-Cog-13 Memory Subscale showed a statistically trending improvement from baseline in the sargramostim group compared to placebo (−1.84, 95% CI: [−3.82, 0.11], p=0.0632) suggesting that the sargramostim treatment effect is primarily on the memory domain (FIG. 3B; Table 4). Unlike the MMSE, the ADAS-COG-13 memory subscale showed no statistically significant improvement within the sargramostim group between baseline and the EOT. The treatment effect was attributable to the worsening of the placebo group. There was no treatment effect for the ADAS-COG-13 Memory Subscore at the 45-day follow up.

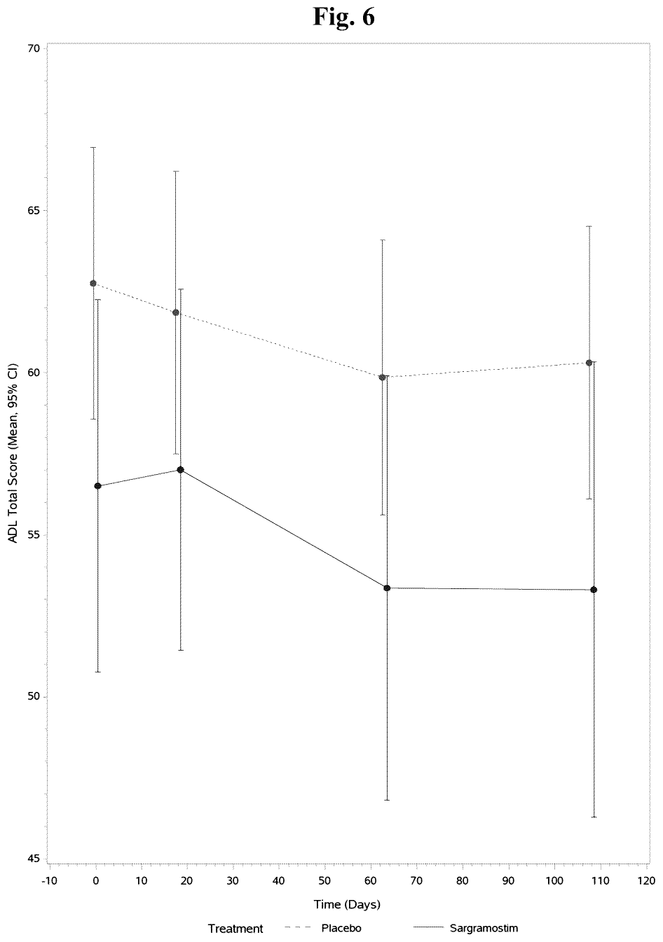

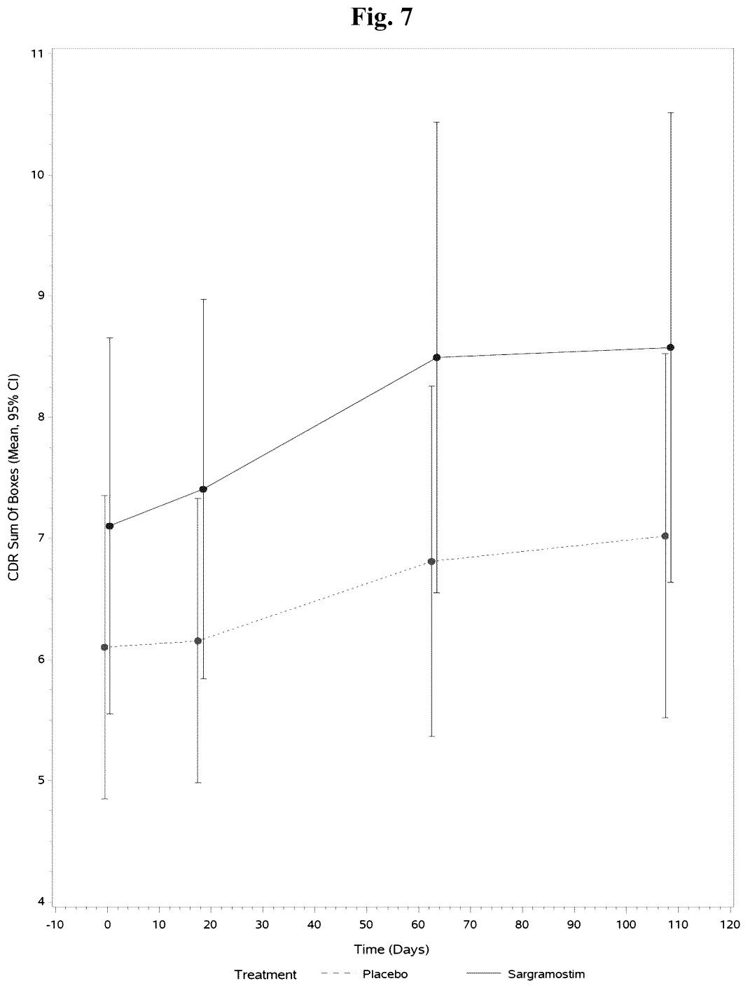

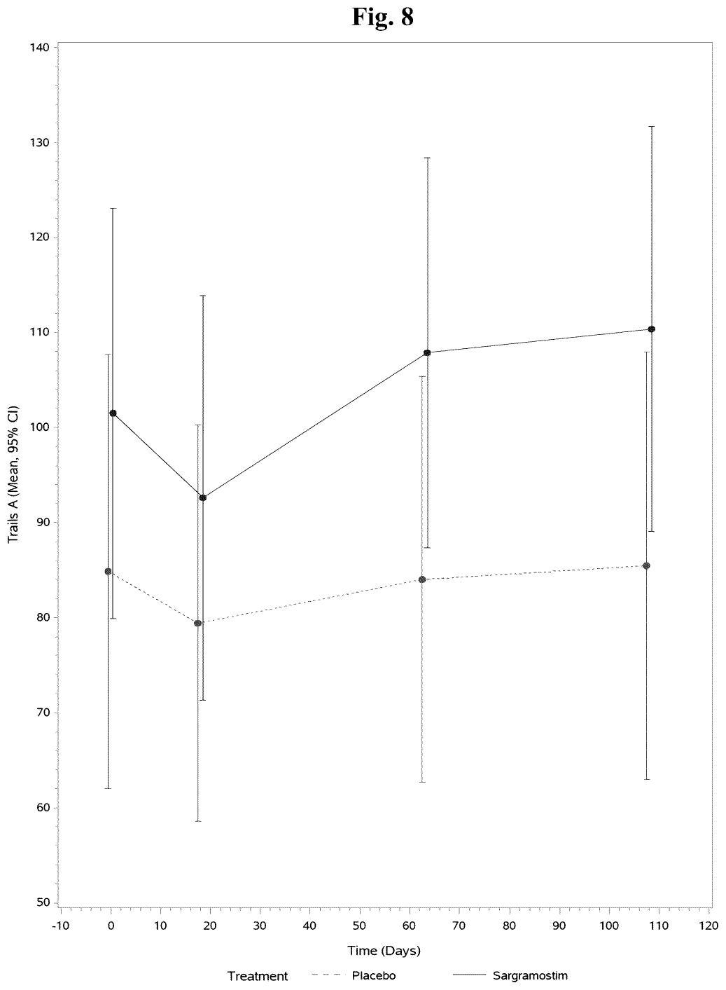

The ADCS-ADL showed a small, non-significant improvement in the sargramostim group at EOT (p=0.3485; FIG. 6; Table 4) but was statistically significantly correlated with MMSE (see below). CDR-SB and TRAILS-A measures showed no statistically significant effects of sargramostim treatment (FIG. 7 and FIG. 8; Table 5).

TABLE 4

| | | | | | | ADAS-Cog-13 | | | |
|---|---|---|---|---|---|---|---|---|---|
| | | MMSE Total | | ADAS Total | | Memory | | ADL Total | |
| | | Score | | Score (13) | | Subscale | | Score | |
| | Time | | Std | | Std | | Std | | Std |
| Treatment | (Categorical) | Mean | Dev | Mean | Dev | Mean | Dev | Mean | Dev |
| Placebo | Baseline | 20.75 | 4.97 | 36.20 | 12.01 | 26.08 | 6.01 | 62.75 | 8.98 |
| | End of Treatment | 20.40 | 5.29 | 36.68 | 11.63 | 27.53 | 6.14 | 61.85 | 9.32 |
| | 1st Follow Up | 19.90 | 5.19 | 36.23 | 9.85 | 27.53 | 6.00 | 59.85 | 9.05 |
| | 2nd Follow Up | 19.40 | 5.47 | 36.85 | 10.24 | 28.15 | 6.33 | 60.30 | 9.00 |
| Sargranoslim | Baseline | 17.10 | 4.58 | 43.21 | 12.45 | 29.76 | 5.49 | 56.50 | 12.30 |
| | End of Treatment | 18.55 | 4.99 | 43.54 | 12.02 | 29.35 | 5.59 | 57.00 | 11.93 |
| | 1st Follow Up | 18.00 | 5.53 | 47.67 | 11.89 | 31.39 | 4.16 | 53.35 | 14.02 |
| | 2nd Follow Up | 17.10 | 5.38 | 45.87 | 13.21 | 29.98 | 5.99 | 53.30 | 15.00 |

TABLE 5

| | | CDR-SB | | TRAILS-A | |
|---|---|---|---|---|---|
| | Time | | Std | | Std |
| Treatment | (Categorical) | Mean | Dev | Mean | Dev |
| Placebo | Baseline | 6.10 | 2.67 | 84.85 | 48.84 |
| | End of Treatment | 5.95 | 2.37 | 79.40 | 44.56 |
| | 1st Follow Up | 6.82 | 3.12 | 84.00 | 45.64 |
| | 2nd Follow Up | 7.03 | 3.27 | 85.45 | 48.10 |
| Sargranoslim | Baseline | 7.10 | 3.32 | 101.50 | 46.17 |
| | End of Treatment | 7.53 | 3.37 | 92.60 | 45.49 |
| | 1st Follow Up | 8.42 | 4.74 | 107.85 | 43.81 |
| | 2nd Follow Up | 8.58 | 4.14 | 110.35 | 45.55 |

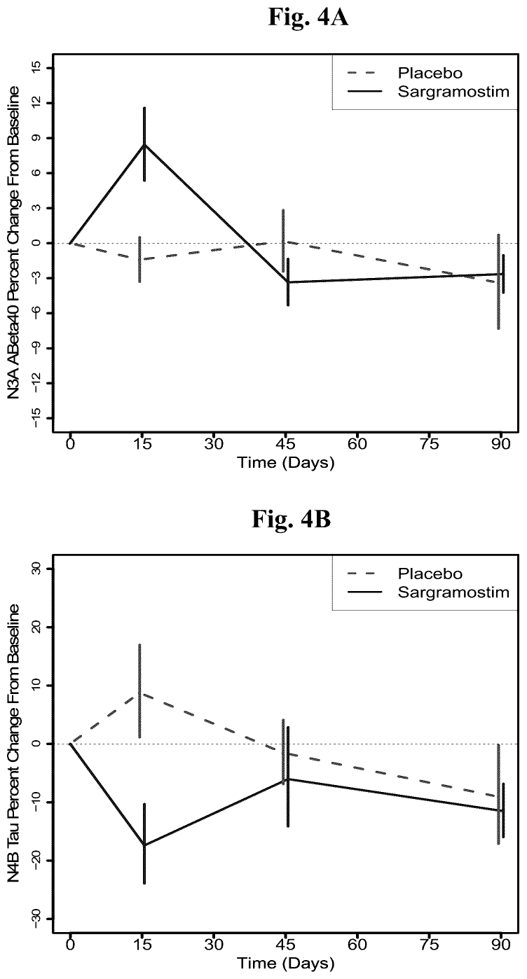

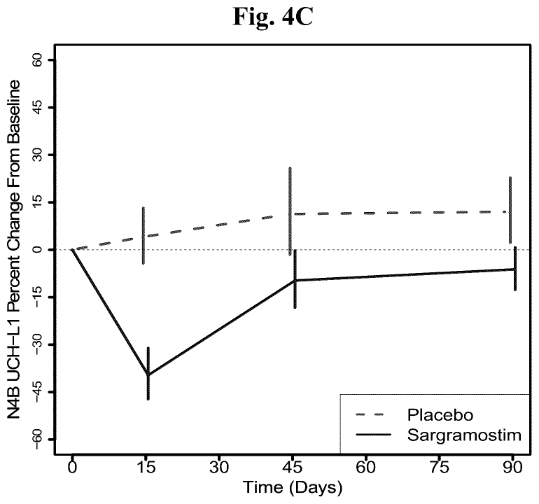

Plasma Biomarkers of AD Neuropathology. To assess changes in brain pathology according to the National Institute on Aging and Alzheimer's Association (NIA-AA) Research Framework, the plasma biomarkers Aβ40, Aβ42, total Tau, UCH-L1, GFAP, and Neurofilament Light (NfL), were measured using an ELISA-based immunoassay. In the sargramostim group at EOT, mean plasma A440 level, which is reduced in AD, showed an 8.4% increase from baseline (p=0.0127), and was 10% higher compared to the placebo-treated group (p=0.0105) (FIG. 4A; Table 6), potentially indicating less sequestration of mon/oligomeric Aβ in the brain. Using the N4PB plate, total tau, whose plasma levels are increased in AD, reflecting both Tau pathology and neurodegeneration, decreased by 17% in the sargramostim-treated group compared to baseline (p=0.0327) and decreased by 24% (p=0.0174) compared to the placebo-treated group baseline change (FIG. 4B; Table 6). At EOT, another independent measure of neurodegeneration, plasma ubiquitin C-terminal hydrolase L1 (UCH-L1), was decreased by 40% in the sargramostim-treated group compared to baseline (p=0.0017)), and by 42% compared to the placebo-treated group change (p=0.0019) (FIG. 4C; Table 6). The N3PA measures of Tau were more variable than the N4PB measures and showed a smaller, non-significant difference between the sargramostim-treated group and the placebo-treated group at EOT.

TABLE 6

| | | Aβ40 | | Total Tau | | UCH-L1 | |
|---|---|---|---|---|---|---|---|
| | | Plasma | | Plasma | | Plasma | |
| | | Levels | | Levels | | Levels | |
| | Time | | Std | | Std | | Std |
| Treatment | (Categorical) | Mean | Dev | Mean | Dev | Mean | Dev |
| Placebo | Baseline | 176.75 | 31.53 | 1.47 | 0.70 | 14.28 | 6.44 |
| | End of | 175.10 | 33.53 | 1.61 | 0.98 | 14.16 | 3.94 |
| | Treatment | | | | | | |
| | 1st Follow Up | 177.56 | 36.02 | 1.44 | 0.60 | 15.38 | 6.63 |
| | 2nd Follow Up | 171.17 | 43.23 | 1.30 | 0.59 | 14.82 | 4.44 |
| Sargrano- | Baseline | 173.83 | 30.32 | 1.39 | 0.55 | 14.65 | 6.69 |
| slim | End of | 188.01 | 41.56 | 1.11 | 0.55 | 8.51 | 4.31 |
| | Treatment | | | | | | |
| | 1st Follow Up | 166.79 | 30.16 | 1.32 | 0.61 | 13.46 | 5.93 |
| | 2nd Follow Up | 168.69 | 25.39 | 1.30 | 0.60 | 14.18 | 6.12 |

Amyloid Imaging. For the second half of the study, amyloid positron emission tomography (amyloid-PET) imaging was used as an inclusion criterion. All 16 participants diagnosed as having mild-to-moderate AD at screening who were then assessed by amyloid-PET imaging were amyloid-positive, validating the clinical inclusion criteria.

The amyloid-PET imaging data at baseline and at HA for the 16 sub-study participants were compared as described above (Amyvid®-PET: two sargramostim-treated, six placebo-treated; ¹¹C-PiB-PET: five sargramostim-treated, three placebo-treated). There were no statistically significant changes in Standardized Uptake Value ratio (SUVr) for each ligand separately (Table 7) or when combined by Centiloid scale conversion (Table 8) or by the method of Properzi.

TABLE 7
Standardized Uptake Value ratio (SUVr)

| | | Time | | Std |
|---|---|---|---|---|
| Ligand | Treatment | (Categorical) | Mean | Dev |
| Amyvid | Sargranoslim | Follow up 1 PET | 1.56 | 0.03 |
| | | Screening PET | 1.74 | 0.14 |
| | Placebo | Follow up 1 PET | 1.60 | 0.14 |
| | | Screening PET | 1.63 | 0.16 |
| PIB | Sargranoslim | Follow up 1 PET | 2.33 | 0.42 |
| | | Screening PET | 2.32 | 0.48 |
| | Placebo | Follow up 1 PET | 2.34 | 0.39 |
| | | Screening PET | 2.34 | 0.37 |

TABLE 8
SUVr - Centiloid Scale Conversion

| | Time | | Std |
|---|---|---|---|
| Treatment | (Categorical) | Mean | Dev |
| Sargranoslim | Follow up 1 PET | 144.98 | 46.53 |
| | Screening PET | 154.20 | 47.85 |
| Placebo | Follow up 1 PET | 133.14 | 37.87 |
| | Screening PET | 137.52 | 38.18 |

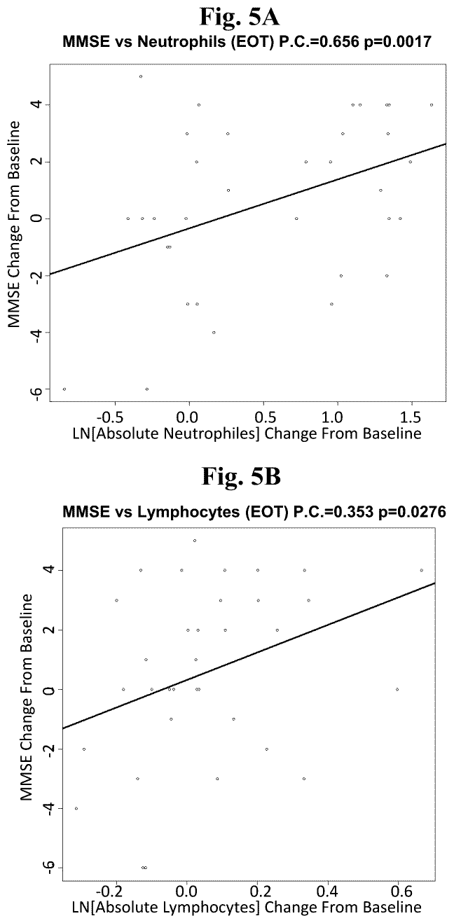

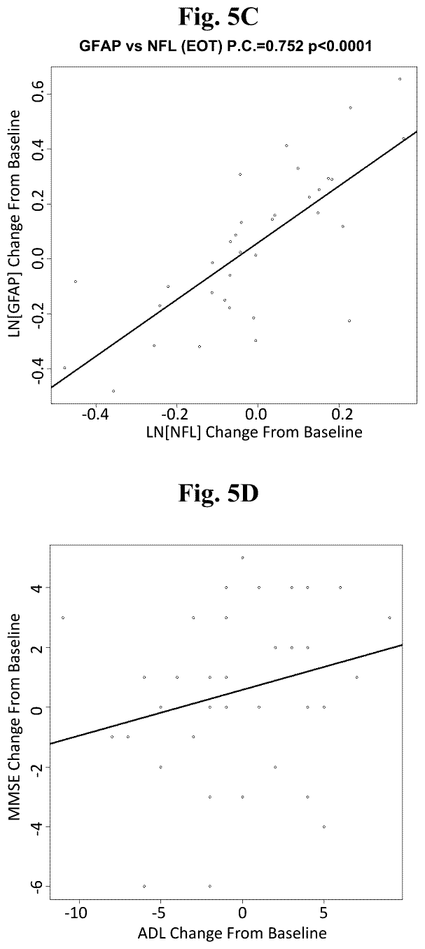

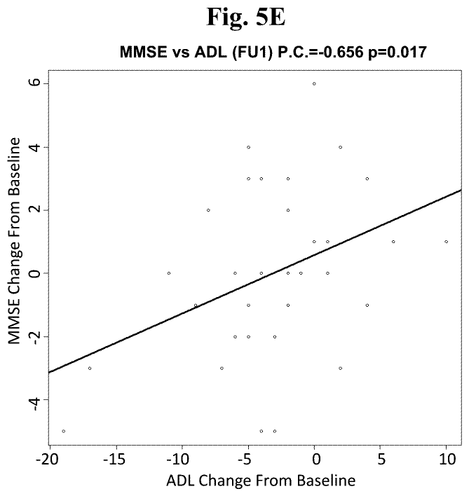

Correlation Analyses. The Pearson correlation between change in MMSE and change in absolute neutrophil counts was statistically significant at the EOT (0.409; p=0.0098) (FIG. 5A). The correlation between change in MMSE and change in absolute lymphocyte counts was also statistically significant at the EOT (0.353; p=0.0276) (FIG. 5B). These results showed that the improvement in cognition measured by MMSE was correlated with (and possibly caused by) the increase in innate immune system stimulation and its downstream effects. There was a very strong correlation between changes in plasma GFAP, a measure of astrocyte activation, and plasma NfL, a measure of neuronal damage, from baseline at all time points for all participants (Pearson coefficient=0.752, 0.693, 0.663 at the EOT, FU1, FU2; all p<0.0001) (FIG. 5C; Tables 9 and 10). Table 9 provides correlations with full participant samples and Table 10 provides correlations with the sargramostim treated participant samples.

TABLE 9

| Variable 1 | Variable 2 | | | | | | |
|---|---|---|---|---|---|---|---|
| (Change From | (Change From | | | Pearson | | Spearman | |
| Baseline) | Baseline) | Time | N | rho | p | rho | p |
| MMSE | ADL | EOT | 40 | 0.26 | 0.1051 | 0.30 | 0.0580 |
| MMSE | ADL | FU 1 | 40 | 0.40 | 0.0116 | 0.38 | 0.0155 |
| MMSE | ADL | FU 2 | 40 | 0.31 | 0.0494 | 0.15 | 0.3622 |
| MMSE | LN[UCH-L1] | EOT | 34 | −0.23 | 0.1972 | −0.30 | 0.0826 |
| MMSE | LN[UCH-L1] | FU 1 | 34 | 0.06 | 0.7261 | 0.03 | 0.8708 |
| MMSE | LN[UCH-L1] | FU 2 | 34 | −0.15 | 0.4039 | −0.22 | 0.2158 |
| LN[GFAP] | LN[NFL] | EOT | 34 | 0.75 | <0.0001 | 0.74 | <0.0001 |
| LN[GFAP] | LN[NFL] | FU 1 | 34 | 0.69 | <0.0001 | 0.79 | <0.0001 |
| LN[GFAP] | LN[NFL] | FU 2 | 34 | 0.66 | <0.0001 | 0.77 | <0.0001 |
| MMSE | LN[Abs Neutrophils] | EOT | 39 | 0.41 | 0.0098 | 0.37 | 0.0189 |
| MMSE | LN[Abs Neutrophils] | FU 1 | 27 | 0.17 | 0.3872 | 0.02 | 0.9221 |
| MMSE | LN[Abs Neutrophils] | FU 2 | 26 | 0.03 | 0.9012 | −0.04 | 0.8533 |
| MMSE | LN[Abs Lymphocytes] | EOT | 39 | 0.35 | 0.0276 | 0.36 | 0.0235 |
| MMSE | LN[Abs Lymphocytes] | FU 1 | 27 | 0.22 | 0.2807 | 0.30 | 0.1232 |
| MMSE | LN[Abs Lymphocytes] | FU 2 | 26 | 0.29 | 0.1494 | 0.16 | 0.4315 |
| MMSE | LN[N4B Tau] | EOT | 34 | −0.19 | 0.2698 | −0.17 | 0.3370 |
| MMSE | LN[N4B Tau] | FU 1 | 34 | 0.00 | 0.9874 | 0.02 | 0.9070 |
| MMSE | LN[N4B Tau] | FU 2 | 34 | −0.07 | 0.7117 | −0.12 | 0.4891 |
| LN[Abeta40] | LN[GFAP] | EOT | 34 | 0.03 | 0.8826 | 0.07 | 0.6910 |
| LN[Abeta40] | LN[GFAP] | FU 1 | 34 | 0.38 | 0.0253 | 0.28 | 0.1091 |
| LN[Abeta40] | LN[GFAP] | FU 2 | 34 | −0.24 | 0.1807 | −0.01 | 0.9733 |
| LN[Abeta40] | LN[N4B Tau] | EOT | 34 | 0.11 | 0.5463 | 0.07 | 0.6973 |
| LN[Abeta40] | LN[N4B Tau] | FU 1 | 34 | −0.04 | 0.8322 | −0.27 | 0.1221 |
| LN[Abeta40] | LN[N4B Tau] | FU 2 | 34 | −0.28 | 0.1134 | −0.29 | 0.0913 |
| MMSE | SUVR (PIB Calibrated) | FU 1 | 16 | −0.34 | 0.2031 | −0.27 | 0.3120 |
| MMSE | LN[Abeta40] | EOT | 34 | 0.19 | 0.2882 | 0.20 | 0.2613 |
| MMSE | LN[Abeta40] | FU 1 | 34 | 0.22 | 0.2162 | 0.26 | 0.1431 |
| MMSE | LN[Abeta40] | FU 2 | 35 | 0.15 | 0.3996 | 0.19 | 0.2729 |
| MMSE | LN[Albumin:Globulin] | EOT | 40 | 0.28 | 0.0805 | 0.33 | 0.0359 |
| MMSE | LN[Albumin:Globulin] | FU 1 | 40 | −0.05 | 0.7440 | −0.07 | 0.6858 |
| MMSE | LN[Albumin:Globulin] | FU 2 | 40 | 0.05 | 0.7630 | 0.02 | 0.8913 |

TABLE 10

| Variable 1 | Variable 2 | | | | | | |
|---|---|---|---|---|---|---|---|
| (Change From | (Change From | | | Pearson | | Spearman | |
| Baseline) | Baseline) | Time | N | rho | p | rho | p |
| MMSE | ADL | EOT | 20 | 0.48 | 0.0340 | 0.50 | 0.0250 |
| MMSE | ADL | FU 1 | 20 | 0.66 | 0.0017 | 0.55 | 0.0112 |
| MMSE | ADL | FU 2 | 20 | 0.43 | 0.0588 | 0.19 | 0.4163 |
| MMSE | LN[UCH-L1] | EOT | 16 | −0.01 | 0.9769 | −0.09 | 0.7515 |
| MMSE | LN[UCH-L1] | FU 1 | 17 | 0.00 | 0.9990 | −0.08 | 0.7470 |
| MMSE | LN[UCH-L1] | FU 2 | 17 | −0.15 | 0.5635 | −0.19 | 0.4676 |
| LN[GFAP] | LN[NFL] | EOT | 16 | 0.92 | <0.0001 | 0.85 | <0.0001 |
| LN[GFAP] | LN[NFL] | FU 1 | 17 | 0.74 | 0.0006 | 0.76 | 0.0004 |
| LN[GFAP] | LN[NFL] | FU 2 | 17 | 0.77 | 0.0003 | 0.78 | 0.0002 |
| MMSE | LN[Abs Neutrophils] | EOT | 20 | 0.24 | 0.2988 | 0.28 | 0.2249 |
| MMSE | LN[Abs Neutrophils] | FU 1 | 13 | −0.05 | 0.8769 | −0.25 | 0.4195 |
| MMSE | LN[Abs Neutrophils] | FU 2 | 11 | 0.43 | 0.1873 | 0.38 | 0.2445 |
| MMSE | LN[Abs Lymphocytes] | EOT | 20 | 0.09 | 0.7158 | 0.06 | 0.8155 |
| MMSE | LN[Abs Lymphocytes] | FU 1 | 13 | 0.38 | 0.2035 | 0.44 | 0.1350 |
| MMSE | LN[Abs Lymphocytes] | FU 2 | 11 | 0.30 | 0.3684 | 0.14 | 0.6910 |
| MMSE | LN[N4B Tau] | EOT | 16 | 0.15 | 0.5752 | 0.43 | 0.0951 |
| MMSE | LN[N4B Tau] | FU 1 | 17 | 0.09 | 0.7232 | 0.05 | 0.8608 |
| MMSE | LN[N4B Tau] | FU 2 | 17 | −0.18 | 0.4804 | −0.26 | 0.3091 |
| LN[Abeta40] | LN[GFAP] | EOT | 16 | 0.03 | 0.9108 | 0.09 | 0.7288 |
| LN[Abeta40] | LN[GFAP] | FU 1 | 17 | 0.14 | 0.5883 | 0.12 | 0.6529 |
| LN[Abeta40] | LN[GFAP] | FU 2 | 17 | −0.01 | 0.9593 | 0.17 | 0.5041 |
| LN[Abeta40] | LN[N4B Tau] | EOT | 16 | 0.58 | 0.0182 | 0.56 | 0.0227 |
| LN[Abeta40] | LN[N4B Tau] | FU 1 | 17 | 0.04 | 0.8804 | −0.04 | 0.8665 |
| LN[Abeta40] | LN[N4B Tau] | FU 2 | 17 | 0.05 | 0.8462 | 0.14 | 0.5798 |
| MMSE | SUVR (PIB Calibrated) | FU 1 | 7 | −0.26 | 0.5770 | −0.32 | 0.4827 |
| MMSE | LN[Abeta40] | EOT | 16 | 0.10 | 0.7201 | 0.13 | 0.6240 |
| MMSE | LN[Abeta40] | FU 1 | 17 | 0.52 | 0.0338 | 0.53 | 0.0271 |
| MMSE | LN[Abeta40] | FU 2 | 18 | 0.36 | 0.1477 | 0.22 | 0.3863 |
| MMSE | LN[Albumin:Globulin] | EOT | 20 | 0.43 | 0.0599 | 0.34 | 0.1381 |
| MMSE | LN[Albumin:Globulin] | FU 1 | 20 | 0.24 | 0.3136 | 0.11 | 0.6512 |
| MMSE | LN[Albumin:Globulin] | FU 2 | 20 | 0.00 | 0.9861 | −0.13 | 0.5864 |

FIG. 5C shows the correlation for the EOT, indicating a likely mechanistic link between neuronal damage and brain glial inflammation in AD and that neuronal damage and gliosis were linked in the brains of participants with mild-to-moderate AD. Within the sargramostim treated group, changes in MMSE were positively correlated with changes in ADL at the EOT (Pearson coefficient=0.476; p=0.034; FIG. 5D) and at HA (0.656; p=0.017; FIG. 5E), the time points that showed a statistically significant treatment effect of sargramostim on MMSE. Thus, the beneficial effect of GM-CSF/sargramostim treatment on MMSE is partially mirrored in another, quite different measure of cognitive function.

The data provided in exemplary Example 1 showed that GM-CSF/sargramostim was safe and well tolerated and provided measurable disease-modifying and memory-enhancing benefits to participants with mild-to-moderate AD.

### Example 2

In another exemplary method, to investigate the interplay of the innate immune system and AD, the effects on AD pathology and behavior of the three hematopoietic colony-stimulating factors (macrophage, granulocyte, and granulocyte-macrophage colony-stimulating factors; M-CSF, G-CSF, and GM-CSF) was studied in transgenic AD mice.

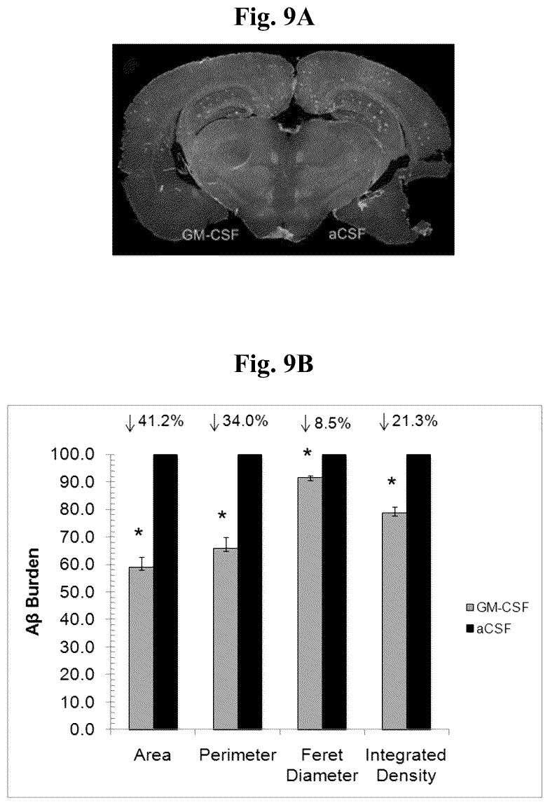

Intrahippocampal injections of colony-stimulating factors. In the first experiment, 5 μg bolus injections of M-CSF, G-CSF, and GM-CSF were injected into the hippocampus of the ipsilateral brain hemisphere with vehicle, artificial cerebrospinal fluid (aCSF), injected into the contralateral hippocampus as a control in aged cognitively-impaired transgenic AD (PS/APP) mice. The mice were then sacrificed one week later and analyzed. While M-CSF injection resulted in significant hyperplasia to the treatment hemisphere and no effect on amyloid deposition, G-CSF showed a modest reduction in amyloid deposition in the injected side. This outcome was confirmed by colleagues using daily peripheral G-CSF injections, which also led to reduced amyloidosis and cognitive deficits in the Radial Arm and Morris Water Mazes. In contrast to the mild G-CSF effect, the GM-CSF injections demonstrated pronounced decreases in amyloid deposition, as compared to control hemispheres (FIG. 9A). Anterior to posterior quantification of amyloid plaques revealed significant reductions within individual mice and overall significant reductions for all plaque parameters measured (FIG. 9B).

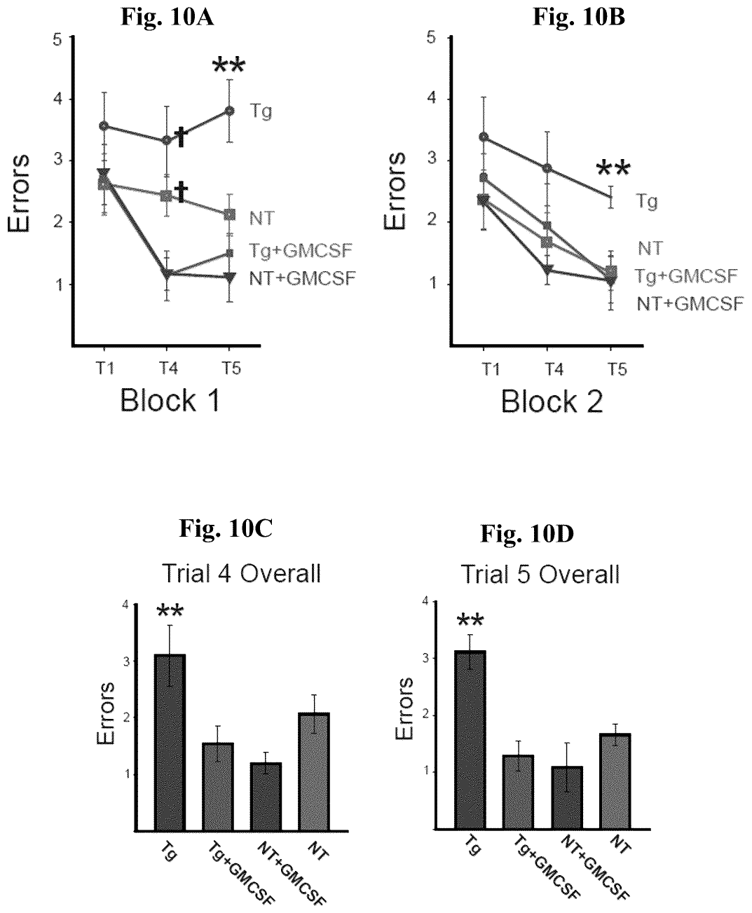

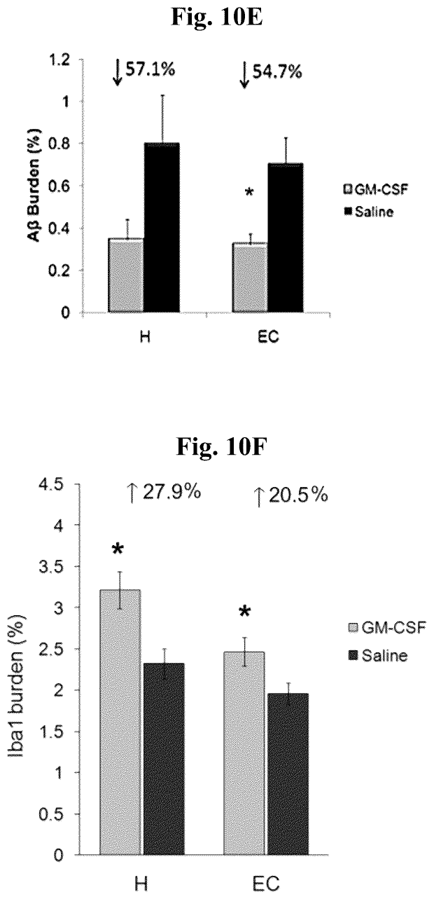

Daily subcutaneous injection of GM-CSF. The effect of subcutaneous GM-CSF injection on AD pathology and cognitive function was next investigated. Prior to GM-CSF treatment, both APPsw transgenic mice (Tg) were first confirmed by RAWM testing to be cognitively-impaired for working memory. Both, the non-transgenic control mice (NT) and the Tg mice were then sub-divided into two cognitively-balanced groups. Twenty days of daily subcutaneous injection of 5 μg of GM-CSF (the most amyloid-reducing CSF in the bolus experiment) were administered to one of the cognitively balanced cohorts of AD mice. RAWM testing post-GM-CSF treatment re-confirmed that Tg control mice were substantially impaired compared to NT control mice. This impairment was evident in individual blocks of testing, as well as over all 4 days of testing (FIGS. 10A-10D). In sharp contrast, GM-CSF-treated Tg mice now performed equally well or better than NT control mice, during individual blocks and overall. Even the GM-CSF-treated NT mice performed as well as, or slightly better than, NT controls (FIG. 10E). An interference cognitive test that assessed the ability of the mice to switch from one water maze to another with different cues, also showed that GM-CSF restored normal cognitive function to the APP and PS/APP animals. Subsequent histological analysis of brains from the APPsw mice of this study revealed that GM-CSF treatment induced large reductions in amyloid burdens within Entorhinal cortex (⬇55%) and the Hippocampus (⬇57%) as compared to control Tg mice (FIG. 10E).

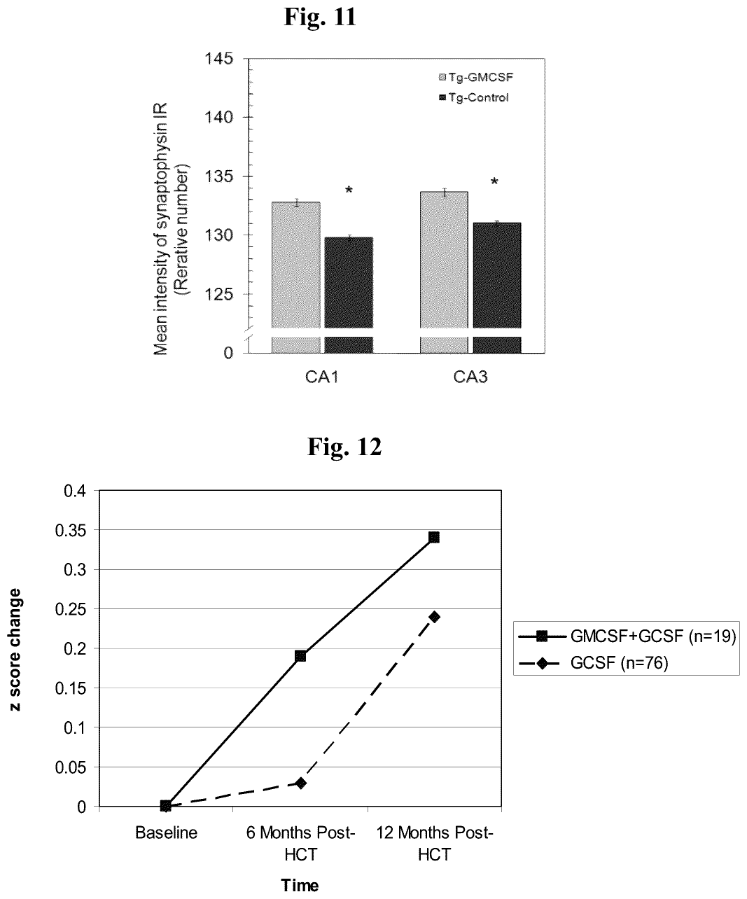

The improved cognitive function and reduced cortical amyloidosis of GM-CSF-treated Tg mice were paralleled by increased microglial density as compared to saline-treated Tg mice (FIG. 10F), implying an augmented ability to bind and remove amyloid deposition. Similarly, the GM-CSF-treated Tg mice demonstrated increased synaptophysin immunoreactivity in both CA1 and CA3 regions (FIG. 11), indicating increased synaptic density in these hippocampal areas.

The data provided in exemplary Example 2 showed that improving cognition can be a new indication and function for GM-CSF.

### Example 3

In another exemplary method, a study was performed assessing cognition in Bone Marrow (Hematopoietic Cell) Transplant (patients, who acquire cognitive deficits from the chemotherapy or irradiation procedures. Their cognitive function was measured before treatments and then 6 months and 12 months after transplant and associated treatment, with a combination of either GM-CSF plus G-CSF or G-CSF alone. The neurological measures used included some of the cognitive measures that are used routinely to assess AD patients.

Total neuropsychological performance z scores (TNP) were calculated by summarizing the cognitive domains of memory, executive functioning (i.e., complex cognition), and attention. Scores indicate change in TNP from pre-transplant baseline. Kruskal-Wallis one-way analyses of variance were conducted at six months and 12 months after baseline assessment and hematopoietic cell transplantation (HCT), using all available data to compare between-group changes in TNP by receipt of GM-CSF. Wilcoxon signed rank tests were conducted using all available data to examine within-group changes in TNP by receipt of GM-CSF. Despite a high level of education (average of 13.89 years), the subjects displayed a statistically significant cognitive deficit at baseline.

The results showed that patients who received GM-CSF plus G-CSF improved their cognitive functions (neuro minus motor features) between base line and six months, whereas subjects receiving only G-CSF did not improve (FIG. 12). This result validates in humans that GM-CSF can improve cognition in an at-risk population using the standard recommended, FDA-approved dosage. This human study also showed that a small number (less than 20) of patients only receiving part GM-CSF at the standard FDA-approved dose was sufficient to reveal that GM-CSF led to improved cognitive measures 6 months after HCT and CSF treatment, compared to the much larger group receiving GM-CSF alone.

### Methods Used in Examples 1-3

The following methodologies were performed in the exemplary methods disclosed in Examples 1-3 as provided herein.

Study Design. The safety and efficacy of GM-CSF (sargramostim/Leukine) in the treatment of Alzheimer's disease was assessed in a phase II, two-center, randomized, three-week-long, double-blind, placebo-controlled, study of sargramostim in patients with mild to moderate Alzheimer's disease (AD). Total planned enrollment was 40 participants, and full enrollment was achieved. The study included a 28-day screening period, 15 treatment days within three weeks, and two follow-up visits at 45-days and 90-days post-treatment. All aspects of the study were conducted in accordance with ethical principles originating from the Declaration of Helsinki and were consistent with good clinical practice and applicable regulatory requirements. Before any study related procedures were conducted, all participants provided written informed consent. Participants with a ¹⁸F florbetapir or ¹¹C-PiB positron emission tomography (PET) scan conducted as part of the study screening process could participate in the ¹¹C-PiB PET imaging substudy. All randomized patients in the studies also had serial magnetic resonance imaging (MM) of the brain.

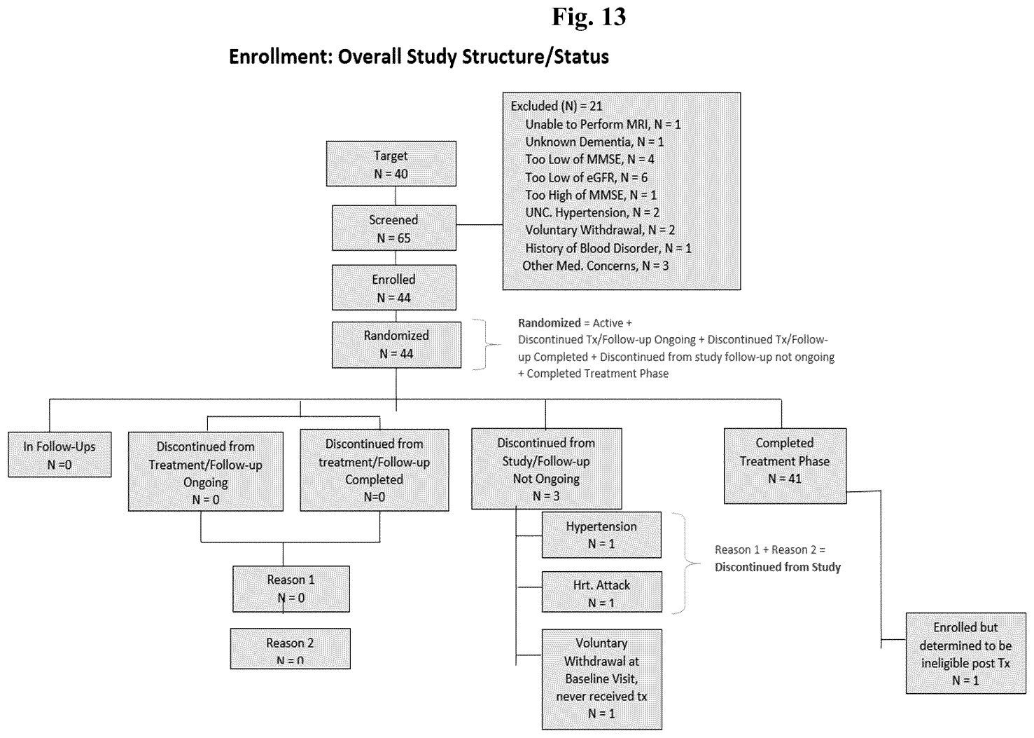

Subjects, Randomization, and Blinding. FIG. 13 provides a schematic of the study design. Once selected, participants were randomized 1:1 to receive a once-daily subcutaneous (SC) dose of sargramostim (250 mcg/m2/day) or placebo. Blinding of the study treatment was achieved using the double-dummy technique. The unblinded pharmacy staff, including the pharmacist manager and technician, prepared the coded syringes for use at clinic. The pharmacy was an independent entity and had the ability to ensure and maintain blinding so that the staff at the clinic did not know which study participants were receiving the study drug or the placebo. All participants, investigators, study staff, and the sponsor were blinded to treatment allocation with limited exceptions as specified in the protocol. The only investigator who was informed of the randomization code was the statistician for the sole purpose of evaluating the results of the trial. Except for the statistician, participants and investigators remain blinded. Study treatment was dispensed during site visits, and eligible participants were injected with the study treatment daily, five days per week, for three weeks, by blinded site nurses or clinicians. Adherence to study treatment was assessed by review of the study source documenting daily injection. All participants who completed the study were adherent.

Study data were collected and managed using REDCap electronic data capture tools, a web-based application designed to support data capture for research studies, providing: 1) an intuitive interface for validated data entry; 2) audit trails for tracking data manipulation and export procedures; 3) automated export procedures for seamless data downloads to common statistical packages; and 4) procedures for importing data from external sources.

Study Drug. Recombinant human GM-CSF is commercially available from Partner Therapeutics (PTx) (a recent spin-off of Genzyme/Sanofi-Aventis) as sargramostim. The FDA-recommended dosage for sargramostim is 250 mcg/m²/day SC for up to 42 days until absolute neutrophil count (ANC) reaches 1,500 cells/mm. Notably, our retrospective study of sargramostim in chemotherapy patients included the recommended FDA-approved dosage (250 mcg/m²/day SC) and found highly significant (p<0.01) cognitive enhancement in patients treated with GM-CSF plus G-CSF compared to those treated with G-CSF alone or those who were untreated. Therefore, in the current trial, treated participants received the full FDA-recommended sargramostim dose. Colorado Scientific Advisory Research Committee to assess our Primary Endpoint of measuring safety. The first injection was administered at the baseline (randomization) visit. For subsequent injections, participants revisited the clinic five days a week for three weeks. Participants returned to the clinic for 45 day and 90 day follow-up assessments.

Blood Biomarkers. CBC with differential Biomarkers: Twice weekly from baseline visit to end-of-treatment and at 45 day and 90 day follow-up visits for each participant, whole blood was collected in an EDTA vacutainer and sent to a contracted laboratory (i.e., Quest) for analyses of complete blood counts (CBC) with differential. Results were sent to the unblinded trial biostatistician to determine sargramostim or placebo group effects on leukocyte populations (i.e., neutrophil, monocyte, and lymphocyte) and albumin/globulin ratios at each visit timepoint.

Plasma Biomarkers: After collection of whole blood in EDTA vacutainers, each vacutainer sample was centrifuged at 1,500×g for 15 min at 22° C. and plasma removed within two hours of collection. Plasma was then centrifuged again at 2,200×g for 10 min at 4° C. to pellet any remaining cells and debris, and then plasma was transferred into 250 μL aliquots and immediately stored at −80° C. All EDTA blood vacutainer tubes and sample aliquots were labeled only with the participant's unique trial ID, the date sample was obtained, and the participant trial visit (i.e., baseline, end-of-treatment, 45 day follow-up, or 90 day follow-up), and all laboratory technicians processing the blood or performing biomarker assays remained blinded to any other identifying participant information or treatment group (i.e., sargramostim or placebo). On days of biomarker analyses, frozen samples were selected in the sample management database, removed from the −80° C. freezer, thawed on ice, and processed according to manufacturer instructions for either MSD (Meso Scale Diagnostics, Inc.) or SIMO (Quanterix) analyses. For each participant, their baseline, end-of-treatment, 45 day follow-up, and 90 day follow-up visit samples were analyzed together on the same assay plate, and each visit's sample was analyzed in triplicate.

For MSD analyses, selected frozen plasma samples were transferred on dry ice to the facilities for thawing, processing, and running the V-PLEX Proinflammatory Panel 1 Human Kit assay, which measures the IFN-γ, IL-1β, IL-2, IL-4, IL-6, IL-8, IL-10, IL-12p70, IL-13, and TNF-α analytes in pg/ml. In preparation for SIMOA analyses using the Neuro 3-Plex A (Aβ40, Aβ42, Tau) and Neuro 4-Plex B (Nf-Light, Tau, GFAP, UCH-L1) assays, selected frozen plasma samples were thawed on ice and transported to the Core for analyses using the Quanterix SR-X Ultra-Sensitive Biomarker Detection System with SIMOA bead technology to measure analytes at fg/ml levels. All MSD and SIMOA results were sent to the trial's unblinded biostatistician for determining any biomarker changes at the end-of-treatment, 45 day and 90 day follow-up visits, as compared to baseline, and for any differences between the sargramostim and placebo groups at the end-of-treatment, 45 day and 90 day follow-up visit time points.

PET. For the last half of the study, PET amyloid imaging was required as an inclusion criterion to assure that the participants indeed had amyloid positive Alzheimer's disease. Ten participants were assessed by florbetapir (i.e., Amyvid) PET imaging (six sargramostim and four placebo) at screening and at 45 days post treatment follow-up visits. When Amyvid was no longer available in the geographic area, two participants determined to be amyloid positive by historical amyloid scan were enrolled, and for eight participants, PET imaging was carried out with Pittsburgh Compound B, synthesized by the Radiology Department both at screening and at the 45-day follow-up visit. All participants diagnosed as having mild-to-moderate AD by the screening physician and then assessed by amyloid PET were indeed amyloid positive.

Amyloid PET images were analyzed by Bioclinica. Freesurfer (version 5.3) was used to obtain regions of interest segmentations on T1 MRI data. Florbetapir and PiB PET data were motion corrected by realignment to the first frame of the raw image file and averaged across frames, smoothed to achieve uniform resolution of 6.5 mm in plane and 7 mm axial FWHM, and registered to the MRI data in T1 native space. The composite cortical Standard Uptake Value Ratio (SUVr) was computed using a grouping of four larger cortical regions including lateral temporal, frontal, parietal (with precuneus), and anterior/posterior cingulate, equally weighted and used as a standard ADNI florbetapir processing method. The whole cerebellum was used as a reference region for SUVr. Centiloid conversion aligns amyloid PET measures across tracers. Bioclinica SUVr pipeline was converted to standard Centiloid scale.

Notably, two participants received Amyvid doses (8.11 mCi and 8.53 mCi, respectively) that were lower than the recommended dosage range for Amyvid (10 mCi+/−10%) at their baseline scans due to delays from having to reboot the PET scanner, and their amyloid-PET imaging data were only used to determine the presence of amyloid to satisfy the inclusion criterion and were not used to quantitate amyloid changes. Thus, the total number of participants who received two usable amyloid-PET scans (i.e., at baseline and at the first follow-up visit) to measure amyloid load was only 16 (Amyvid: two sargramostim-treated and six placebo-treated; PiB: five sargramostim-treated and three placebo-treated). The average injected dose for florbetapir was 10.39 mCi (SD 0.38) at baseline and 10.19 mCi (SD 0.39) at follow-up, and the average injected dose for PiB was 14.84 mCi (SD 0.37) at baseline and 14.68 mCi (SD 0.49) at follow-up.

MRI. Brain MRI was performed for each participant at baseline, end of treatment (15 days), and first follow-up (45 days after the end of treatment). MRI was performed without intravenous gadolinium and included 3D T1, axial T2, axial FLAIR, diffusion-weighted images (DWI), and susceptibility weighted images (SWI). Each MRI study was visually reviewed and formally interpreted by a board-certified neuroradiologist to ensure no structural or physiological abnormalities that would preclude enrollment and to assess the potential development of adverse changes in the brain following treatment (i.e., Amyloid Related Imaging Abnormalities [ARIAS]), such as vasogenic edema and microhemorrhage. The presence of microhemorrhage was assessed on SWI, an imaging technique highly sensitive to hemosiderin and other blood products. No significant changes in gross parenchymal volume, white matter disease, or baseline microhemorrhage was observed for any participant over the MRI scans. Quantitative morphometry of T1 images was performed for the purposes of PET analysis, as described in the above section. Radiological MRI reports would have been shared with potential subjects' primary care physician if they were deemed clinically relevant, but none were.

Cognitive Tests. One or more of the following cognitive tests were performed where indicated:

MUSE (Mini-Mental State Exam.) Education corrected Folstein Mini-Mental State Exam is a standard cognitive assessment used by all ADRCs including the FADRC to identify and monitor AD subjects.

ADAS-cog (Alzheimer's Disease Assessment Scale-cognitive subscale). ADAS was designed to measure the severity of the most important symptoms of AD. Its subscale ADAS-cog is the most popular cognitive testing instrument used in clinical trials of nootropics (drugs or agents that improve cognitive function). It consists of 11 tasks measuring the disturbances of memory, language, praxis, attention and other cognitive abilities which are often referred to as the core symptoms of AD.

ADCS/ADL (Alzheimer's Disease Cooperative Study Activities of Daily Living Inventory). ADCS-ADL is a caregiver rated questionnaire of 23 items, with possible scores over a range of 0-78, where 78 implies full functioning with no impairment. The ADCS-ADL assesses functional capacity across a wide spectrum of severity and will be the primary tool for collecting ADL data.

TRAILS A. Psychomotor speed will be assessed by the Trail Making Test-A, a timed test in which subjects must connect a series of numbers randomly placed on a page.

TRAILS B. Executive function will be assessed using Trail Making Test-B, a sensitive test of cognitive flexibility and psychomotor speed in which subjects must connect a series of alternating numbers and letters placed randomly on a page.

Mohs Cancellation Task. Psychomotor speed and cognitive vigilance will be assessed using Mohs Cancellation Task, a timed task involving cancellation of target stimuli in a larger array of distractors.

CDR (Clinical Dementia Rating). The CDR is a caregiver and subject based interview to assess changes in domains such as memory, orientation, judgment and problem solving, community affairs, home and hobbies, and personal care. Each domain is rated as 0 (no dementia), 0.5 (uncertain dementia), 1 (mild dementia), 2 (moderate dementia), or 3 (severe dementia).

Data Safety Monitoring Plan and Protection of Human Subjects. Physical exams were performed at screening, baseline, end of treatment, and at follow-up visits (45 and 90 days post-treatment). Vital signs, injection site review, and adverse event (AE) monitoring were performed by the clinic nurse on each treatment day. Electrocardiograms (ECGs) were performed at screening. MRIs were performed at screening, and at end of treatment. As recommended by the current sargramostim product information, we obtained two CBCs with differential weekly, and at follow-ups (45 and 90 days post-treatment). Sargramostim dosage was reduced by half in only one subject whose total ANC rose above 20,000/mm³. CMP was performed at screening, baseline, weekly during the treatment period, at end of treatment, and at follow-ups (45 and 90 days post-treatment). A summary schedule of all study events is presented in Table 11.

TABLE 11
STUDY SCHEDULE

| | VISITS | | | | | | | | | | | | | | | | | |
|---|---|---|---|---|---|---|---|---|---|---|---|---|---|---|---|---|---|---|
| | | | | | | | | | | | | | | | | | Follow | End of |
| | Screen | SCHEDULE TREATMENT PHASE | | | | | | | | | | | | | | | up 1 | study |
| | DAYS | | | | | | | | | | | | | | | | | |
| | | | | | | | | | | | | | | | | | 45 days | 90 days |
| | 7-28 | 1 | 2 | 3 | 4 | 5 | 6 | 7 | 8 | 9 | 10 | 11 | 12 | 13 | 14 | 15 | post | post |
| Informed consent | X | | | | | | | | | | | | | | | | | |
| Pregnancy test | X | | | | | | | | | | | | | | | | | |
| GFR | X | | | | | | | | | | | | | | | | | |
| MMSE | X | X | | | | | | | | | | | | | | X | X | X |
| Medical history | X | | | | | | | | | | | | | | | | | |
| concomitant meds | X | X | X | X | X | X | X | X | X | X | X | X | X | X | X | X | X | X |
| Adverse events | | X | X | X | X | X | X | X | X | X | X | X | X | X | X | X | X | X |
| Vital signs | X | X | X | X | X | X | X | X | X | X | X | X | X | X | X | X | X | X |
| Physical exam | X | | | | | | | | | | | | | | | X | X | X |
| Injection site review | | X | X | X | X | X | X | X | X | X | X | X | X | X | X | X | | |
| ECG | X | | | | | | | | | | | | | | | | | |
| MRI | X | | | | | | | | | | | | | | | X | X | |
| PET | X | | | | | | | | | | | | | | | | X | |
| LABS | | | | | | | | | | | | | | | | | | |
| Biomarkers | | X | | | | | | | | | | | | | | X | X | X |
| CBC + diff | X | X | | | X | | X | | | X | | X | | | | X | X | X |
| CMP | X | | | | X | | | | | X | | | | | | X | X | X |
| Cognitive testing | | X | | | | | | | | | | | | | | X | X | X |
| Study drug | | X | X | X | X | X | X | X | X | X | X | X | X | X | X | X | | |
| injections | | | | | | | | | | | | | | | | | | |

Outcome Measures

Safety: The primary outcome in this study was to evaluate safety and tolerability of sargramostim in participants with mild-to-moderate AD. Safety and tolerability were evaluated using the following key assessments: spontaneously reported AEs, laboratory tests, vital signs, physical examinations, including neurological examinations, and MM to examine for any possible amyloid related imaging abnormalities. The independent DSMB reviewed the participant data at the end of each cohort of ten, to assess and assure clinical safety, including assessing evidence of serious cognitive decline or ARIAs such as microhemorrhages or vasogenic edema attributable to the study drug.

Efficacy: The secondary, exploratory endpoint was to evaluate the potential efficacy of sargramostim (250 mcg/m2/day) compared with placebo in improving the cognition is participants with mild-to-moderate AD, as measured by change in score from baseline to the end of the double-blind, placebo-controlled period by the Mini-Mental State Examination (MMSE) and the 13-item Alzheimer Disease Assessment Scale-cognitive subscale (ADAS-Cog₁₃). The MMSE is a commonly used, brief cognitive assessment used within both clinical trials and clinical practice to identify and monitor individuals with AD. The assessment has a score range of 0-30 points, with higher scores indicating better performance. The ADAS-Cog₁₃ measures severity of impairment in various cognitive domains (memory, language, orientation, praxis, and executive functioning). The assessment has a score range of 0 to 85 points, with higher scores indicating worse performance. Both scales were analyzed as a continuous measure.

Exploratory endpoints included efficacy evaluations of sargramostim vs placebo on the change from baseline to the end of the placebo-controlled treatment periods on the following functional, cognitive, and clinical outcomes: Alzheimer's Disease Cooperative Study-Activities of Daily Living Inventory (ADCS-ADL), Clinical Dementia Rating-Sum of Boxes (CDR-SB), and Trail Making Test A.

Imaging biomarker endpoints included evaluation of the effect of sargramostim on Medial Temporal Lobe Atrophy (MTA) via serial MRIs, and amyloid load via serial ¹¹C-PiB PET scans. Blood biomarkers include amyloid, Tau, and neurodegeneration markers (Aβ1-42 and Aβ1-40, Nf-Light®, Tau, GFAP, UCH-L1) and various inflammation-associated cytokines and chemokines (IFN-γ, IL-1β, IL-2, IL-4, IL-6, IL-8, IL-10, IL-12p70, IL-13, and TNF-α).

Statistical Analyses

Overview. The primary endpoint, safety, was assessed in the safety population (patients who received >1 dose of study treatment). All participants who received at least one dose of the study treatment post-randomization were included in the safety analyses. Analyses of treatment-emergent adverse events, laboratory results, vital signs, and MRI scans were performed. The samples sizes in the study were calculated to yield greater than 60% power to detect a medium effect size of 0.5 for the cognitive tests and PET.

Efficacy analyses (secondary and exploratory endpoints) were conducted in the per-protocol population, which included all randomized patients who completed the treatment period. The secondary endpoint outcome measures, change in score from baseline until the end of the placebo-controlled period, and the follow-up visits for the MMSE and the ADAS-Cog₁₃, were analyzed using a mixed model of repeated measures (MMRM) analysis, with the change from baseline at each scheduled post baseline visit acting as the dependent variable. Other efficacy outcomes including the ADCS-ADL, CDR-SB, and Trail Making Test A were analyzed in the same manner.

An interim analysis that included data analysis by the statistician only was performed and reviewed by select senior study personnel during the conduct of the study, but without unblinding. Statistical analyses were performed using a software program (SAS Institute Inc). All hypotheses were tested at a 2-sided 0.05 significance level. No adjustments for multiple comparisons were made.

Statistical Analyses Details. Scale outcomes were analyzed with longitudinal regression models. Time was treated categorically, and each time×treatment combination mean was free to vary. Unstructured matrices by treatment were used to model the covariance among repeated measures for maximum flexibility. Denominator degrees of freedom were calculated using the Satterthwaite method. Linear combinations of the time×treatment combination means were constructed and tested.

To compare the treatment means at baseline, we estimated and tested the contrast of the sargramostim group baseline mean minus the placebo group baseline mean. Within-treatment-group changes were estimated and tested by subtracting the treatment group baseline mean from each of the later time point means for the same treatment. The treatment effects were estimated and tested by subtracting the within-placebo-group changes from the within-sargramostim-group changes, equivalent to the time×treatment interaction. The treatment group difference at baseline was not a factor because the within-treatment-group changes from their respective baselines were compared. F tests for a treatment effect at any time point, or at either of the two follow-up visit time points (45 and 90 days after the end of treatment), were performed as well. Models with the baseline outcome as covariate were fit as well, and the outcome's change from baseline was modeled.

Available change scores, between screening and the first follow-up visit, of SUVr and Centiloid measures of amyloid, were compared between treatment groups with two sample T-tests. Two different ligands were used during the study: ¹⁸F florbetapir (Amyvid) and PiB. The amyloid analyses were performed both within each ligand and for the two combined. For the combined ligand analysis, either the Centiloid scale or a cumulative distribution method was used to transform the SUVr measurements for one ligand type into the other (FIGS. 14A-14C). Estimates, 95% confidence intervals, and p values are presented. Univariate alpha=0.05.

Tests were not adjusted for multiple testing because of limited power, the number of outcomes, and the exploratory goal of this aspect of the trial. Data were analyzed on an available case basis. Statistics were calculated with SAS 9.4 and R.

All of the COMPOSITIONS and METHODS disclosed and claimed herein can be made and executed without undue experimentation in light of the present disclosure. While the COMPOSITIONS and METHODS have been described in terms of preferred embodiments, it will be apparent to those of skill in the art that variation can be applied to the COMPOSITIONS and METHODS and in the steps or in the sequence of steps of the METHODS described herein without departing from the concept, spirit and scope of the invention. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of improving cognition in a subject having a neurodegenerative condition, the method comprising
administering to the subject a composition comprising granulocyte macrophage colony stimulating factor (GM-CSF);
analyzing at least one blood sample from the subject comprising at least one of before, during, and after administering the GM-CSF composition to the subject for leukocytes in the blood sample, and optionally, analyzing the at least one blood sample for at least one of 1) concentration of one or more inflammatory markers, 2) concentration of one or more neurodegenerative condition-linked biomarkers, and 3) ratio of albumin to globulin in the blood sample, and
adjusting treatment of the subject based on at least one of the absolute number of leukocytes and optionally, at least one of 1) the concentration of the one or more inflammatory markers, 2) concentration of the one or more neurodegenerative condition-linked biomarkers, and 3) the ratio of albumin to globulin in the at least one blood sample.

2. The method according to claim 1, wherein the leukocytes comprise at least one of neutrophils, lymphocytes and monocytes.

3. The method according to claim 1 or 2, wherein the leukocytes comprise lymphocytes and monocytes and wherein treatment is ceased for a predetermined period of time when the number of lymphocytes in the blood sample is an absolute count of about 15 and the number of monocytes in the blood sample is an absolute count of about 30.

4. The method according to claim 3, wherein GM-CSF continues until the number of lymphocytes in the blood sample is an absolute count of about 15 and the number of monocytes in the blood sample is an absolute count of about 30.

5. The method according to claim 1, wherein when absolute leukocyte numbers after GM-CSF treatment reaches greater than 20,000 per milliliter of the at least one blood sample then GM-CSF concentration in the treatment is reduced or treatment is discontinued for a period of time.

6. The method according to any one of claims 1-5, further comprising applying at least one cognition assessment test to the subject at least one of before, during, and after administering the GM-CSF to the subject.

7. The method according to claim 6, wherein the at least one cognition assessment test comprises a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), or a combination thereof.

8. The method according to any one of claims 1-7, further comprising analyzing at least one blood sample from the subject comprising at least one of before, during, and after administering the GM-CSF composition to the subject for the presence of one or more neurodegenerative condition-linked biomarkers in the blood sample.

9. The method according to claim 8, wherein the one or more neurodegenerative condition-linked biomarkers comprises Amyloid beta, Tau, ubiquitin C-terminal hydrolase L1 (UCH-L1), or a combination thereof.

10. The method according to claim 9, wherein analyzing the at least one blood sample for the at least one of the concentration of one or more neurodegenerative condition-linked biomarkers further comprises continuing a GM-CSF treatment regimen when concentration of Tau in the blood sample is about 10% but at least less than 30% compared to a baseline measurement of Tau, wherein the baseline measurement of Tau is measured in blood of the subject before administering the GM-CSF treatment regimen to the subject.

11. The method according to claim 9, wherein analyzing the at least one blood sample for the at least one of the concentration of one or more neurodegenerative condition-linked biomarkers further comprises continuing a GM-CSF treatment regimen when concentration of UCH-L1 in the blood sample is about 10% but at least less than 30% compared to a baseline measurement of UCH-L1, wherein the baseline measurement of UCH-L1 is measured in the blood before administering the GM-CSF treatment regimen to the subject.

12. The method according to claim 9, wherein GM-CSF treatment regimen continues until both Tau and UCH-L1 concentration in the blood samples are about 10% or less compared to a baseline measurement of both Tau and UCH-L1 in blood of the subject, wherein the baseline measurement of both Tau and UCH-L1 is measured in blood of the subject before administering the GM-CSF treatment regimen to the subject.

13. The method according to claim 9, wherein GM-CSF treatment is halted if at least one of Tau and UCH-L1 concentration in the blood samples are 30% or greater compared to a baseline measurement of both Tau and UCH-L1, wherein the baseline measurement of at least one of the Tau and UCH-L1 is measured in blood of the subject before administering the GM-CSF treatment regimen to the subject.

14. The method according to any one of claims 1-14, further comprising analyzing at least one blood sample from the subject comprising at least one of before, during, and after administering the GM-CSF composition to the subject for the ratio of albumin to globulin in the at least one blood sample.

15. The method according to claim 14, wherein analyzing the at least one blood sample for the ratio of albumin to globulin further comprises continuing a GM-CSF treatment regimen when ratio of albumin to globulin of the at least one blood sample is 8% or more below the baseline ratio of albumin to globulin, wherein the baseline ratio of albumin to globulin is measured in blood of the subject before administering the GM-CSF treatment regimen to the subject.

16. The method according to claim 14, wherein GM-CSF treatment is halted if the ratio of albumin to globulin in the blood is below a baseline ratio of albumin to globulin by at least 9% or more, wherein the baseline ratio of albumin to globulin is measured in the blood before administering the GM-CSF treatment regimen to the subject.

17. The method according to claim 16, wherein GM-CSF treatment is restarted when the ratio of albumin to globulin in the blood returns to a percentage below the baseline ratio of albumin to globulin by at least 5% or less.

18. The method according to claim 1, wherein the GM-CSF is sargramostim.

19. The method according to claim 1, wherein administering GM-CSF to the subject comprises administering GM-CSF to the subject for at least five days per week for up to three weeks.

20. The method according to any one of claims 1-19, further comprising analyzing the at least one blood sample from the subject comprising at least one of before, during and after administering the GM-CSF composition to the subject for the presence of one or more inflammatory markers in the blood sample.

21. The method according to claim 20, wherein the at least one pro-inflammatory marker comprises a cytokine.

22. The method according to claim 21, wherein the cytokine comprises one or more of interleukin (IL)-2, IL-6, IL-8, IL-10, Tumor Necrosis Factor (TNF)-alpha, or other pro-inflammatory cytokine.

23. The method according to any one of claims 1-22, wherein GM-CSF treatment is continued when cognitive state of the subject is improved after administration of GM-CSF for at least five days per week for up to three weeks.

24. The method according to any one of claims 1-23, wherein GM-CSF is administered to the subject by at least one of by parenteral route or orally.

25. The method according to any one of claims 1-24, wherein the subject is a human subject.

26. The method according any one of claims 1-25, wherein the neurodegenerative condition comprises one or more of Alzheimer's disease, frontotemporal dementia, vascular dementia, viral infection, or a combination thereof.

27. A method for administering granulocyte macrophage colony stimulating factor (GM-CSF) to a subject having a neurodegenerative condition, the method comprising:
a) performing at least one cognition assessment test on the subject as a baseline cognitive measurement;
b) collecting at least one baseline blood sample from the subject:
c) measuring at least one of an absolute number of leukocytes, concentration of at least one inflammatory cytokine, concentration of at least one neurodegenerative condition biomarker, a ratio of albumin to globulin in the at least one baseline blood sample;
d) administering GM-CSF for at least five days per week for up to three weeks to the subject;
e) performing the at least one additional cognition assessment test to the subject after administering GM-CSF in d);
f) collecting at least one additional blood sample from the subject after administering GM-CSF in d) and optionally, repeating c);
h) comparing data obtained in the baseline of the at least one additional cognition assessment test and the at least one additional blood sample; and
i) adjusting GM-CSF treatment in the subject as needed based on the comparison data.

28. The method according to claim 27, wherein the at least one cognition assessment test comprises a Mini-Mental State Exam (MMSE), an Alzheimer's Disease Assessment Scale-cognitive subscale (ADAS-cog), an Alzheimer's Disease Cooperative Study Activities of Daily Living Inventory (ADCS/ADL), or a combination thereof.

29. The method according to any one of claim 27 or 28, wherein the neurodegenerative condition comprises mild Alzheimer's disease, moderate stage Alzheimer's disease, or severe Alzheimer's disease.

30. A kit for determining a treatment regimen for administering granulocyte macrophage colony stimulating factor (GM-CSF) to a subject, the kit comprising one or more devices for collecting a blood sample from a subject and determining one or more of 1) leukocytes levels; 2) concentration of one or more neurodegenerative condition biomarkers; 3) concentration of cytokines, in the at least one blood sample; or any combination thereof and; a GM-CSF composition.

31. The kit according to claim 30, wherein the kit further comprises:
(i) at least one container for the at least one blood sample; and/or
(ii) one or more reagents for determining protein levels of one or more neurodegenerative condition biomarkers from the blood sample.
